SEQUENCE LISTING (22 sequences)

SEQ ID NO: 1 - RNA, 23 nt, Homo sapiens
  source 1..23: mol_type = genomic RNA
ttaatgctaatcgtgataggggt

SEQ ID NO: 2 - DNA, 23 bp, synthetic construct
  source 1..23: mol_type = other DNA
acccctatcacgattagcattaa

SEQ ID NO: 3 - DNA, 23 bp, Homo sapiens
  source 1..23: mol_type = genomic DNA
ttaatgctaatcgtgataggggt

SEQ ID NO: 4 - DNA, 23 bp, synthetic construct
  source 1..23: mol_type = other DNA
ttaatgctaatcgtgataggggc

SEQ ID NO: 5 - DNA, 23 bp, synthetic construct
  source 1..23: mol_type = other DNA
ttaatgctaatcgtgataggtgt

SEQ ID NO: 6 - DNA, 23 bp, synthetic construct
  source 1..23: mol_type = other DNA
ttaatgctaatcgtgatatgggt

SEQ ID NO: 7 - DNA, 23 bp, synthetic construct
  source 1..23: mol_type = other DNA
ttaatgctaatcgtgacaggggt

SEQ ID NO: 8 - DNA, 23 bp, synthetic construct
  source 1..23: mol_type = other DNA
ttaatgctaatcgttataggggt

SEQ ID NO: 9 - DNA, 23 bp, synthetic construct
  source 1..23: mol_type = other DNA
ttaatgctaatcttgataggggt

SEQ ID NO: 10 - DNA, 23 bp, synthetic construct
  source 1..23: mol_type = other DNA
ttaatgctaaccgtgataggggt

SEQ ID NO: 11 - DNA, 23 bp, synthetic construct
  source 1..23: mol_type = other DNA
ttaatgcttatcgtgataggggt

SEQ ID NO: 12 - DNA, 23 bp, synthetic construct
  source 1..23: mol_type = other DNA
ttaatgttaatcgtgataggggt

SEQ ID NO: 13 - DNA, 23 bp, synthetic construct
  source 1..23: mol_type = other DNA
ttaacgctaatcgtgataggggt

SEQ ID NO: 14 - DNA, 23 bp, synthetic construct
  source 1..23: mol_type = other DNA
ttaatgctaatcgtgatagggac

SEQ ID NO: 15 - DNA, 23 bp, synthetic construct
  source 1..23: mol_type = other DNA
ttaatgctaatcgtcctaggggt

SEQ ID NO: 16 - DNA, 23 bp, synthetic construct
  source 1..23: mol_type = other DNA
ttaatgctcctcgtgataggggt

SEQ ID NO: 17 - DNA, 23 bp, synthetic construct
  source 1..23: mol_type = other DNA
ttcctgctaatcgtgataggggt

SEQ ID NO: 18 - DNA, 23 bp, synthetic construct
  source 1..23: mol_type = other DNA
ccaatgctaatcgtgataggggt

SEQ ID NO: 19 - DNA, 23 bp, synthetic construct
  source 1..23: mol_type = other DNA
tattgtggattcgcgagttaaga

SEQ ID NO: 20 - DNA, 22 bp, synthetic construct
  source 1..22: mol_type = other DNA
tagcttatcagactgatgttga

SEQ ID NO: 21 - DNA, 22 bp, synthetic construct
  source 1..22: mol_type = other DNA
taacattgtctggtaaagatgg

SEQ ID NO: 22 - DNA, 21 bp, synthetic construct
  source 1..21: mol_type = other DNA
tgagatgaagcactgtagctc

(19) United States
(12) Patent Application Publication
(10) Pub. No.: US 2024/0384343 A1
(43) Pub. Date: Nov. 21, 2024

(54) GRAPHENE OXIDE-BASED FLUORESCENT SENSOR FOR BIOMOLECULAR DETECTION

(71) Applicant: City University of Hong Kong, Hong Kong (HK)

(72) Inventors: Pik Kwan Peggy LO, Hong Kong (HK); Pan LI, Hong Kong (HK); Ling Sum LIU, Hong Kong (HK); Fei WANG, Dongguan (CN); Hoi Man LEUNG, Hong Kong (HK)

(21) Appl. No.: 18/632,333

(22) Filed: Apr. 11, 2024

Related U.S. Application Data
(60) Provisional application No. 63/502,404, filed on May 15, 2023.

(51) Int. Cl. C12Q 1/6876; C12Q 1/6818

(52) U.S. Cl. CPC C12Q 1/6876; C12Q 1/6818; C12Q 2600/178

(57) ABSTRACT

The present invention provides a stable and reliable biosensor (GO-TNA) composed of chemically modified TNA capture probes onto graphene oxide (GO) for detecting and imaging of target nucleic acids in vitro and in vivo, discriminating single nucleobase mismatch and monitoring target miRNA dynamic changes. The TNA capture probes in the invention brings significant improvement by 1000 times in the detection limit when compared to TNA probes itself. This detection platform is facile and cost-effective to prepare, which are critical advantages for real-time analysis, and thus brings new opportunities in the field of disease diagnosis for rapid and accurate detecting and imaging of a variety of disease-related nucleic acid molecules at the in vivo level. Apart from healthcare, they may also lead to potential applications in the areas of environmental analysis and food safety through detection of pesticides, antibiotics, or even foodborne pathogenic agents.

### CROSS-REFERENCE TO RELATED APPLICATION

The present application claims priority from a U.S. provisional patent application Ser. No. 63/502,404 filed May 15, 2023, and the disclosures of which are incorporated by reference in its entirety.

The sequence listing file under the name “P2749US01_sequence_listing.xml” submitted in ST.26 XML file format with a file size of 20.0 KB created on Apr. 11, 2024 is incorporated herein by reference.

### FIELD OF THE INVENTION

The present invention generally relates to the field of biosensors, specifically the development of a stable and reliable biosensor. The biosensor is composed of chemically modified TNA capture probes onto graphene oxide (GO) for detecting and imaging of target nucleic acids in vitro and in vivo. It has the ability to discriminate single nucleobase mismatch and monitor target miRNA dynamic changes.

### BACKGROUND OF THE INVENTION

The variation of nucleotide sequence in the human genome, for instance, single nucleotide mutation, can cause a number of human genetic diseases such as β thalassemia, thalassemia, Huntington's disease, color-blindness, and cystic fibrosis, etc. Indeed, the number of genetic disorders is significantly increasing in recent decades. Apart from the diseases of genetic origin, the single nucleotide mutation also creates susceptibility towards some critical illnesses and neurological disorders including stroke, cancers, Parkinson's disease and Alzheimer's disease. Consequently, highly sensitive and sequence-specific detection of target nucleic acids is of the utmost importance for reliable and early diagnosis of disorders and for subsequent monitoring of disease treatment to improve disease prognosis and slow down disease progression. The development of nucleic acid-based biosensors, where their detection principle mostly relies on nucleic acid hybridization or target interactions with aptamers, is growing rapidly because they have shown potential applications in clinical analysis of a variety of human diseases.

So far, natural DNA has been widely used as a biorecognition probe for nucleic acid sensing, but it shows some limitations such as poor reproducibility, nuclease-induced enzymatic degradation, binding interference and reduced bioactivity on solid substrates. Enzymes, including nucleases, have evolved to specifically recognize and degrade DNA/RNA, particularly targeting natural nucleic acids. These enzymes identify specific structural features, such as the presence of a 2′-hydroxyl group on the deoxyribose sugar of natural nucleic acids. This structural characteristic reduces the biostability of DNA/RNA, making them less resistant to the hydrolytic action of enzymes found in serum or biological environments. In addition, complex biological samples like serum, plasma, or tissue contain various biomolecules that can interfere with the binding of the DNA probe to the target miRNA. Consequently, this interference, combined with the poor biostability of DNA probes, can negatively impact the accuracy and reliability of miRNA detection. To tackle these challenges, scientists have been actively exploring the potential of xeno nucleic acids (XNAs) as alternative capture and sensing probes for accurate nucleic acid detection. XNAs are chemically modified analogs of natural DNA/RNA that possess distinct properties, pro-viding numerous specific advantages and unique attributes. These include chemical diversity, stability, modified base pairing, biological inertness, functional diversity, and biocompatibility. By leveraging these characteristics, XNAs offer a compelling solution to overcome these issues. For example, chemical modifications of the phosphate linkage, sugar and non-ribose moieties of nucleic acids would improve their properties in terms of binding affinity to RNAs/DNAs, nuclease and protease resistance, toxicity, thermal stability, etc. So far, phosphorothioate (PS) modified oligonucleotides, locked nucleic acid (LNA), peptide nucleic acid (PNA) and morpholino (MO) have been studied and used as stable capture probes for biosensing of target DNAs or miRNAs in cultured cells, blood and bacteria samples, producing optical, mechanical or electrochemical signals as detection outputs.

The recent advancements and breakthroughs in sugar-modified XNAs have inspired researchers to investigate the potential of un-natural TNA as a versatile biomaterial. TNA, a synthetic polymer, has garnered attention due to its resemblance to RNA and the unique properties it offers. This RNA-like nature makes TNA an intriguing candidate for various applications in the field of biomaterials. TNA is made up of a backbone repeating unnatural 4-carbon threose sugar with phosphodiester linkages taking place at the 2′-and 3′-vicinal positions of the threofuranose ring (see below). The pioneer's work by Eschenmoser indicated that TNA is able to form a stable duplex Watson-Crick antiparallel structure with its complementary partner. Compared to natural nucleic acids, we have shown that synthetic TNAs did not induce pathological changes, and severe functional and structural damage in the renal systems. TNAs also exhibited substantial cellular uptake and high stability in pH extremes, human blood serum, fetal bovine serum and even under storage in various buffer solutions at room temperature for a long period. Compared to natural DNA and phosphorothioate DNA, the same sequence of TNA exhibited a stronger binding affinity towards its complementary RNA. The AMBER force-field optimized structures of TNA exhibited a better match to natural RNAs than DNAs. Specifically, a slightly more un-wound, ladder-like arrangement between the synthetic TNA strand and the RNA strand is obtained. This observation can be attributed to the presence of oxygen at the 2′ position of both RNA and TNA sugar moieties. The steric hindrance resulting from the clash be-tween the oxygen atom at the 2′ position and the oxygen at the 3′ position renders the canonical B-form helical conformation un-favorable. Apart from being an aptamer and a catalyst, TNA become an active biomaterial and alternative to traditional antisense oligonucleotides for suppressing target gene expression and inhibiting tumor growth in vivo with no harmful effect. More recently, we used TNA itself as a building block to construct a miRNA biosensor in a partial duplexed structure (pd-TNA) for real-time, intracellular nucleic acid detection and imaging. Its detection mechanism relied on toehold-mediated strand displacement strategy to dissociate the fluorophore-labeled TNA reporter strand from the pd-TNA biosensor after the miRNA target fully bound to the quencher-labeled TNA recognition strand, resulting in a recovery of fluorescence signals. However, its limit of detection is found to be 2.5 nM which is comparable to other XNA systems but is still not satisfactory for monitoring of miRNA dynamic changes in various cellular states. Thus far, no in vivo study of these unnatural synthetic capture probes for nucleic acid detection has been reported.

Chemical structure of TNA:

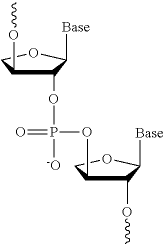

Rapid, accurate and sensitive detection of nucleic acids in complex biological matrices plays a significant role but remains a big challenge. To enhance signal output and reliability, scientists put a lot of effort into exploiting the power of nanomaterials as innovative biosensing platforms with fascinating roles at biological interfaces. Gold nanoparticles, silicon nanowires, paper-based electrode, glassy carbon electrode, graphene oxides (GOs), and graphene field-effect transistor have been widely used as substrates for immobilization or conjugation of XNA-based capture probes. Among them, GO has been reported as an effective quenching platform for nucleic acid detection to improve the sensitivity and reduce the background noise due to its excellent aqueous solubility, amphiphilicity, biocompatibility, surface functionalizability, and fluorescence quenching capability. The GO-based biosensing platforms depend on the preferential x-x interactions and hydrogen bonding of GO surface with single-stranded oligonucleotides as functional recognition elements over double-stranded oligonucleotides or high-ordered DNA structures. The quenched fluorescence is restored as a detection signal after targeting nucleic acids hybridized with the sensing probes, followed by dissociation from GO's surface.

Unfortunately, it is found that a natural nucleic acid-based sensing probe is not able to effectively distinguish one or two nucleotides deletion, resulting in poor specificity and selectivity. Although researchers incorporated structurally modified unnatural nucleic acid probes such as peptide nucleic (PNA) or locked nucleic acid (LNA) with GO for improving sensitivity and selectivity towards miRNA detection. However, concerns of rapid clearance and high toxicity limit their biomedical applications in disease diagnosis and therapy after targeting to miRNAs.

The development of natural nucleic acid-based sensing probes for microRNAs (miRNAs) detection in living systems is of significance in investigating miRNA-regulated signaling pathways and guiding the discase diagnosis and prognosis, particularly, cancers. However, their development into viable diagnosis systems has faced challenges with regard to low sensitivity and throughput, time consuming, high experimental costs and analytical complexity. The present invention addresses the above issues.

### SUMMARY OF THE INVENTION

Provided herewith is a high-specificity, high-selectivity and high-biocompatibility GO-TNA biosensor capable of detecting oncogenic miRNAs in vitro and in vivo, discriminating single nucleobase mismatch and monitoring target miRNA dynamic changes.

In a first aspect of the present invention, a high-specificity, high-selectivity and high-biocompatibility nucleobase mismatch-distinguishing RNA biosensory nanoplatform is provided, comprising a threose nucleic acid (TNA) sensing probe and a fluorescence quencher, wherein the TNA sensing probe is nucleobase-engineered to be antisense to a target RNA transcript.

In a first embodiment of the first aspect of the present invention, the TNA sensing probe further comprises a fluorophore tag. In a further embodiment, the specific fluorophore tag is Cy3.

In another embodiment, the fluorescence quencher of the high-specificity, high-selectivity and high-biocompatibility nucleobase mismatch-distinguishing RNA biosensory nanoplatform is graphite oxide.

A method of detecting and/or imaging a target RNA transcript through the aforementioned high-specificity, high-selectivity and high-biocompatibility nucleobase mismatch-distinguishing RNA biosensory nanoplatform is also provided herewith.

The method of detecting and/or imaging a target RNA transcript comprises: (i) synthesizing a TNA sensing probe through nucleobase engineering, such that the TNA sensing probe is antisense to the target RNA transcript; (ii) introducing a fluorescence dye tag to the TNA sensing probe; (iii) binding the antisense capture sequence of the TNA sensing probe to the target RNA transcript; (iv) forming a double-stranded TNA-RNA duplex structure; and (v) dissociating the TNA-RNA duplex structure from the fluorescence quencher surface.

In an embodiment of the above method, the target RNA transcript further comprises a microRNA miRNA-155.

In a further embodiment, the aforementioned method is specifically designed for the detection and imaging of oncogenic microRNAs.

### BRIEF DESCRIPTION OF THE DRAWINGS

Embodiments of the invention are described in more details hereinafter with reference to the drawings, in which:

FIG. 1 shows a schematic diagram of the design and working principle for the GO-TNA biosensor in living cells.

FIGS. 2A and 2B show the MALDI-TOF spectra for Cy3TNA-155 TNA of m/z calcd 7133.380; found 7142.965 [M⁺] (FIG. 2A) and Cy3scrTNA of m/z calcd 7133.380; found 7143.138 [M⁺] (FIG. 2B) respectively.

FIG. 3A shows the fluorescence intensity of Cy3TNA-155 at 562 nm after addition of GO at various time points. FIG. 3B Fluorescence spectrum of Cy3TNA-155 after the addition of different amount of 0.1 mg/mL GO (from 8.2 to 9.2 μL). (Excitation: 532 nm, and emission: 562 nm.)

FIGS. 4A and 4B depict the fluorescence spectra of GO-Cy3TNA-155 (100 nM) detection platform after addition of complementary miRNA-155 and DNA-155 respectively at different time points.

FIG. 5A depicts the fluorescence intensities of GO-Cy3TNA-155 (100 nM) detection platform at 562 nm after addition of different concentrations of non-complementary oligonucleotide, DNA-155 strands and miRNA-155. FIG. 5B shows the fluorescence recovery effects of GO-Cy3TNA-155 (100 nM) after addition of 100 nM of miRNA-155 and DNA-155 in Tris-HCl buffer as a function of incubation time. (Excitation: 532 nm, and maximum emission collected: 562 nm.) FIGS. 5C and 5D respectively show the fluorescence intensities of GO-Cy3TNA-155 (100 nM) detection platform after addition of complementary DNA-155, non-complementary oligonucleotide and control DNA strands including D-21, D-141, D-143 (FIG. 5C), and one nucleotide mismatch (red color) and two nucleotide mismatch (blue color) DNA-155 oligonucleotides at different positions (FIG. 5D). (Excitation: 532 nm, and emission: 562 nm.)

FIGS. 6A to 6C respectively depicts the fluorescence spectra of GO-Cy3TNA-155 (100 nM) detection platform after addition of complementary miRNA-155 (FIG. 6A), complementary DNA-155 (FIG. 6B) and non-complementary oligonucleotide (FIG. 6C) at different concentrations. (Excitation: 532 nm.)

FIG. 7 depicts the fluorescence spectra of GO-Cy3TNA-155 (100 nM) detection platform before and after addition of complementary DNA-155, non-complementary oligonucleotide, and control DNA strands including D-21, D-141, D-143 at concentration of 100 nM. (Excitation: 532 nm)

FIG. 8 depicts the fluorescence spectra of GO-Cy3TNA-155 (100 nM) detection platform after addition of complementary DNA-155, 1-mismatched DNA-155 and 2-mismatched DNA-155 in different positions with the concentration of 100 nM. (Excitation: 532 nm)

FIG. 9 depicts the fluorescence spectra of Cy3TNA-155 strand after storage for half a year.

FIGS. 10A and 10B depicts calibration curves of the GO-Cy3TNA-155 detection platform after adding complementary DNA-155 from concentrations of 0 to 100 nM, and the limit of detection (LOD) is found to be 33.881±0.15 μM (FIG. 10A); and complementary miRNA-155 from concentrations of 0 to 100 nM, and the limit of detection (LOD) is found to be 28.335±0.12 pM (FIG. 10B).

FIG. 11A depicts the fluorescence intensity of detection platforms immobilized with Cy3TNA-155 or Cy3DNA-155 with and without FBS treatment. FIG. 11B depicts the relative cell viability of four different cancer cells after incubating with GO-Cy3TNA-155 detection platforms for 24 h.

FIG. 12 depicts the fluorescence spectra of detection platforms immobilized with Cy3TNA-155 or Cy3DNA-155 with and without FBS treatment.

FIG. 13A depicts the denaturing PAGE analysis of Cy-labelled TNA and the corresponding Cy3-labeled DNA incubated with 10% FBS solution at various time points. FIG. 13B illustrates the relationship between the amount of remaining TNA or DNA at different time points.

FIGS. 14A to 14C shows confocal fluorescence images of BT-549, MCF-7 and MDA-MB-468 cells after treating with GO-Cy3scrTNA and GO-Cy3TNA-155 detection platforms for 8 hours respectively. (Scale bar=50 μm)

FIG. 15 depicts the result of RT-qPCR analysis of miRNA-155 expression in different cell lines.

FIG. 16A depicts RT-qPCR analysis of miRNA-155 expression in MDA-MB-231 cells. FIG. 16B depicts the fluorescence intensity of MDA-MB-231 cells. FIG. 16C shows the confocal fluorescence images of MDA-MB-231 cells after treating with GO-Cy3TNA-155 detection platform for 8 h in the absence and presence of inhibitors or mimics. FIG. 16D shows the confocal fluorescence images of MDA-MB-231 cells after treating with GO-Cy3TNA-155 detection platform at different time points. (Scale bar=50 μm)

FIGS. 17A and 17B depicts in vivo detection of miRNAs in MDA-MB-231 tumor model. In vivo fluorescence imaging (FIG. 17A) and quantified fluorescence intensities of MDA-MB-231 tumor-bearing mice (FIG. 17B) after intratumoral injection of GO-Cy3TNA155 and GO-Cy3scrTNAs at different time points are shown. FIG. 17C shows the confocal fluorescence imaging of tumor tissues isolated after intratumoral injection of GO-Cy3TNA155 and GO-Cy3scrTNAs for 4 hours. (Scale bar, 50 μm)

FIGS. 18A to 18C respectively illustrate the fluorescence intensities of detection platform with capture probes in GO-TNA systems using Cy3TNA-20 (FIG. 18A), Cy3TNA-21 (FIG. 18B) and Cy3TNA-141 (FIG. 18C), after the addition of complementary DNA pairs and N-CPM-ON.

### DETAILED DESCRIPTION

Provided herewith is a high-specificity, high-selectivity and high-biocompatibility nucleobase mismatch-distinguishing biosensory nanoplatform based on GO-TNA, capable of detecting oncogenic miRNAs in vitro and in vivo, discriminating single nucleobase mismatch and monitoring target miRNA dynamic changes.

Specifically, the GO-TNA biosensory platform is constructed by loading the TNA sensing probes (Cy3TNA-155) with well-designed sequence, which complement to the sequences of target miRNA-155, through physical adsorption.

Subsequently, its sensitivity, selectivity and biocompatibility are examined and compared with the GO platform loaded with DNA capture probes. Single mismatching recognition of GO-TNA are fully evaluated by mutating the nucleotide of the target nucleic acid at different positions.

Afterwards, its capability of dynamic monitoring of miRNAs expression level in different cancer cell lines are examined and compared. Finally, the capacity of GO-TNA biosensors for in vivo detection of target nucleic acids are investigated using tumor xenograft models.

The stable TNA-based GO sensing platform may be used for precise detection, identification and quantification of a large variety of targets such as disease-related nucleic acid molecules, pesticides, antibiotics, nucleic acids, proteins, peptides, metabolites and foodborne pathogenic agents by simply utilizing different sequences of TNA capture probes.

The TNA-GO biosensors are constructed by immobilizing fluorophore-labeled, single-stranded TNA (SSTNA) as recognizing probe onto graphite oxide as fluorescence quencher. The present invention can be used to in situ detect and image a wide range of oncogenic miRNAs which are overexpressed in various human cancers by simply changing the sequences of TNAs. The sequence of TNAs can be designed to be antisense to any target microRNA which is a key regulatory molecule overexpressed in various cancer cells for tumor initiation and progression.

Multiple features of the invention are novel in comparison with current technology, as listed below.

Advantageously, the present invention structurally modifies unnatural nucleic acid-based GO platform as a sensitive and stable biosensor for both in vitro and in vivo detection.

The TNA-GO biosensors in the present invention adopt a versatile design and fabrication to achieve biological stability in a cost-effective manner for real-time detecting and monitoring of the expression level of miRNA targets in living systems.

The miRNA detection duration of this TNA-GO biosensor is shorter than some of previously reported nucleic acid-based probes, resulting in an improvement in the detection efficiency by the TNA-GO biosensor of the present invention.

A comparable detection limit is calculated at picomolar scale for the antisense probe of the present invention, enabling the TNA-GO biosensor a significantly higher detection and imaging resolution.

The TNA-GO biosensor of the present invention also has excellent selectivity and capability in distinguishing base mismatches in target RNA molecules (specific discrimination of highly similar targets), allowing a higher accuracy of detection in comparison with other similar technologies.

The present invention has superior resistance to enzymatic degradation over natural nucleic-based probes, and the excellent storage stability enables the use of TNA-GO biosensor for long-term studies in a broad range of biological samples.

The TNA-GO biosensor of the present invention is also advantageously designed to optimize cellular uptake by different cancer cells without the need of transfection, thereby ensuring efficient uptake by living cells with negligible cytotoxicity for dynamic real-time monitoring target miRNAs and differentiate the distinct target miRNA expression levels in various cancer cell lines.

As compared to PNA or LNA-based recognizing probes, by advantageously developing a TNA-based probe, the biosensor of the present invention has no sequence limitation, allowing optimum flexibility to engineer the probe to correspond to different target RNA transcripts.

When the TNA probe of the present invention pairs up with a nucleic acid transcript, the heteroduplex formed (e.g. TNA/DNA and TNA/RNA), as compared with the homoduplexes formed by natural nucleic acids (DNA/DNA, DNA/RNA and RNA/RNA), possesses greater thermodynamic stability, thereby enabling the use of TNA sensing probe which can be shorted than the equivalent DNA or RNA probes.

As such, the present invention enriches nanomaterial development and contributes significantly to a new generation of high-throughput and low-cost diagnostic platforms for clinical applications.

As set forth below, GO-TNA biosensors, TNA capture probes and GO-based fluorescence quencher are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

The variation of nucleotide sequence in the human genome, for instance, single nucleotide mutation, can cause a number of human genetic discases. Thus, rapid, accurate and sensitive detection of nucleic acids in complex biological matrices plays a significant role but remains a big challenge. Natural deoxyribonucleic acids (DNAs) have been widely used as biorecognition probes but they show some limitations including poor specificity and reproducibility, nuclease-induced enzymatic degradation, and reduced bioactivity on solid substrates.

The present invention addresses the problem by providing a graphene oxide-based biosensor for detecting and imaging nucleic acid. The biosensor is composed of unnatural synthetic thrcose nucleic acids and acts as a capture probe. In particular, the biosensor is composed of fluorophore-labeled, sequence-specific TNAs as recognizing probes and graphite oxide (GO) as a fluorescence quencher for rapid miRNA detection. The TNA recognizing probe is designed to be antisense to a target RNA transcript via pair pairing. The TNA-based GO biosensor depends on the preferential x-x interactions and hydrogen binding of GO surface with fluorescence-labeled, ssTNA recognizing strands, resulting in effective quenching of fluorescence signals. In the presence of RNA targets, the antisense capture sequence of the recognizing probe binds to targeted transcripts, forming double-stranded TNA-RNA duplex structure.

These target binding events give rise to the dissociation of the TNA-RNA complexes from GO's surface and then followed by “turning-on” of the fluorescence as a detection signal. The extent of fluorescence enhancement is quantifiably related to the target RNA expression level in living systems. Interestingly, this TNA-based GO biosensor is highly specific and selective towards target miRNAs and is able to distinguish one or two nucleotides deletion.

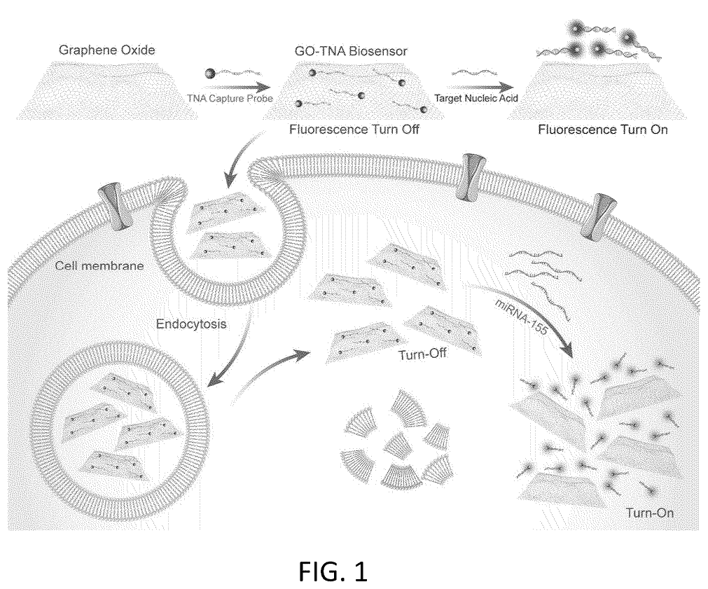

A schematic of the design principle for the GO-TNA biosensor is present in FIG. 1. Briefly, the nucleic acid detection nanoplatform is composed of Cy3-labelled, sequence-specific TNAs as recognizing probe and GO as a fluorescence quencher. The sequence of Cy3-labeled recognizing probe (Cy3TNA-155) complement to the sequence of target miR-155 is designed. Scramble TNA oligonucleotides (Cy3scrTNAs) are also designed with same number and composition of nucleotides but in different orders as control probes. These sequence-designed TNA oligonucleotides are synthesized on an automatic nucleic acid synthesizer according to the well-established solid-phase synthetic protocols.

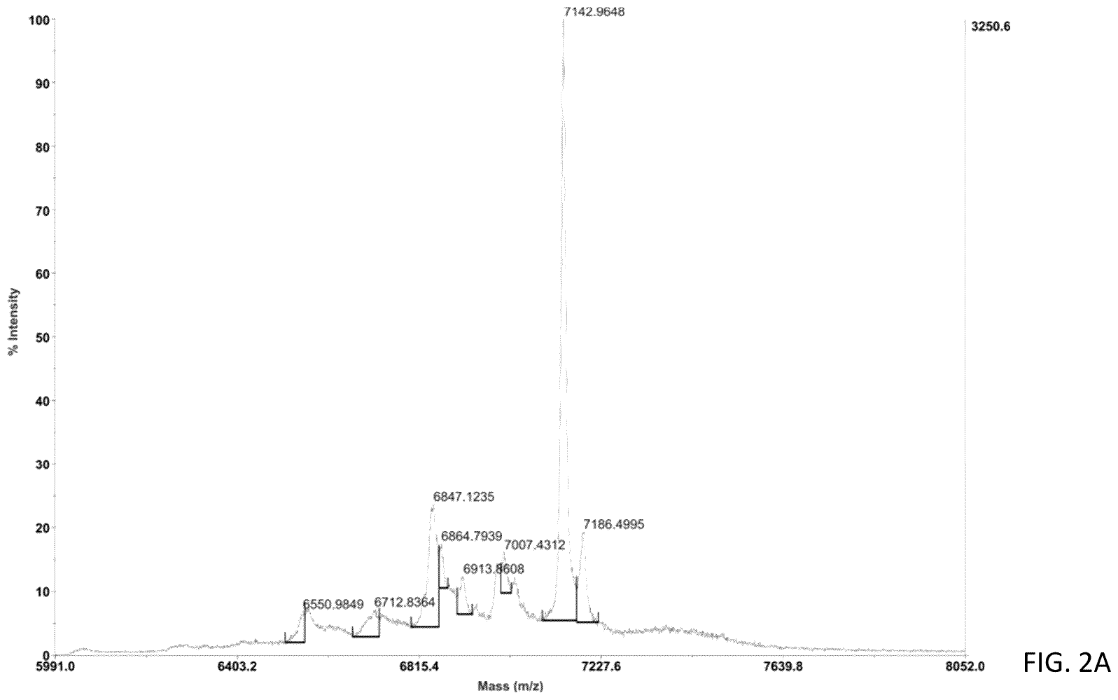

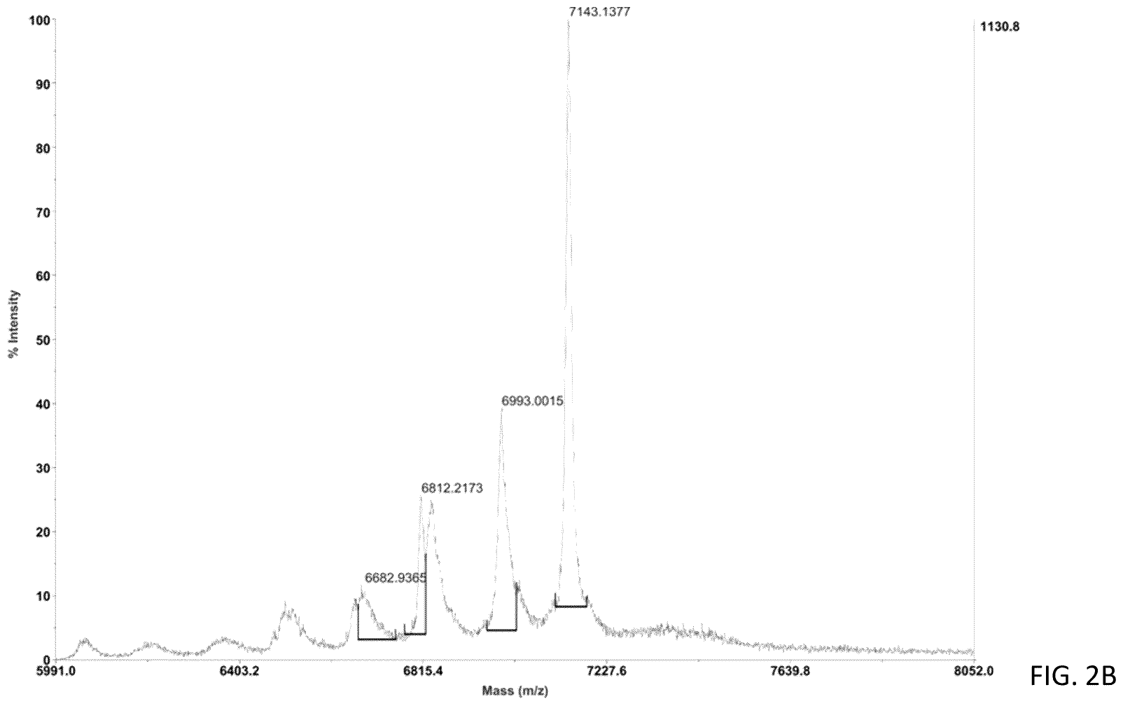

Followed by denaturing polyacrylamide gel electrophoresis (PAGE) purification, two Cy3-labeled sequence-specific TNA strands are generated and characterized by high resolution MALDI-TOF mass spectrometry studies (FIG. 2). The sequences of the probes and tested oligonucleotides are shown in Table 1 below.

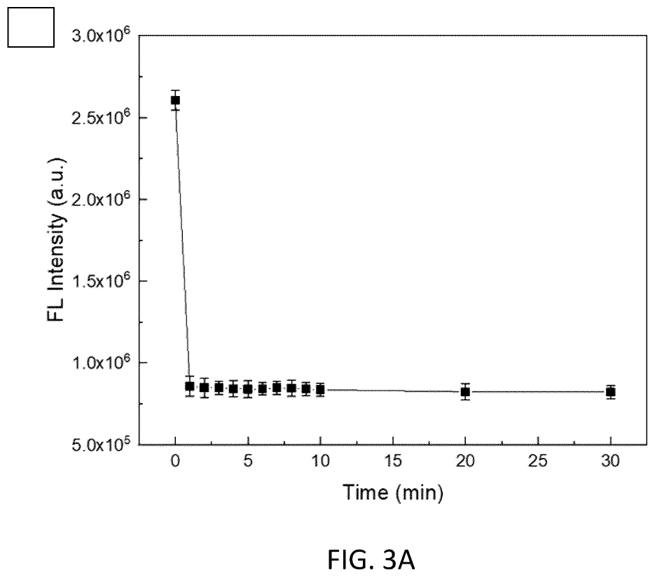

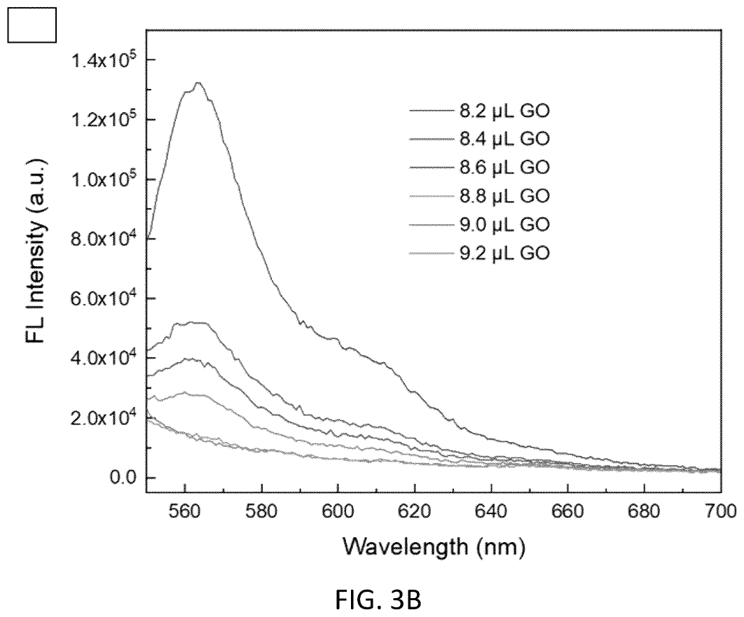

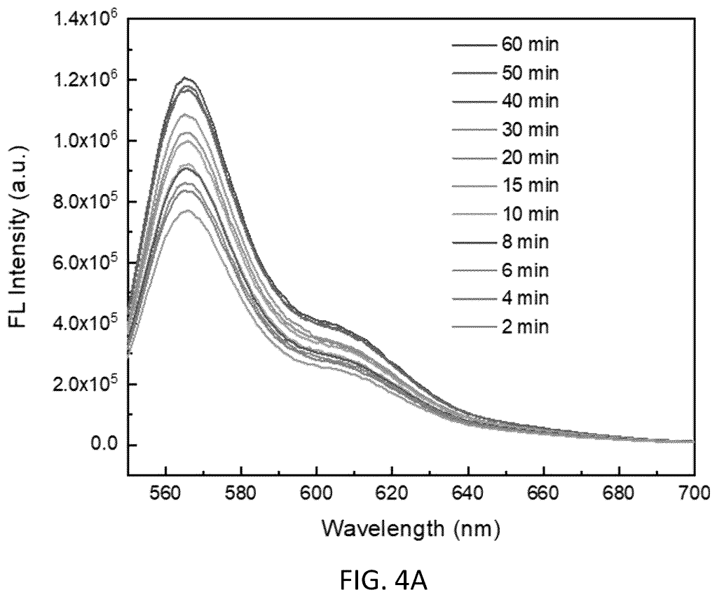

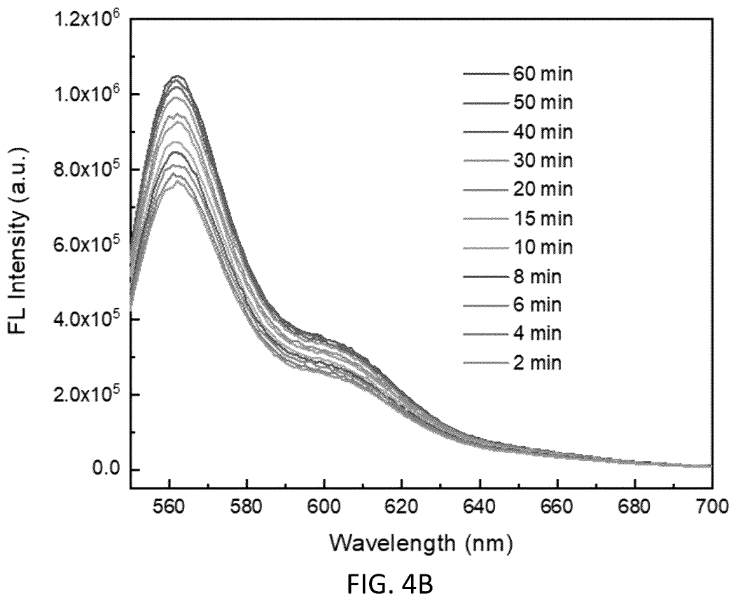

The successful formation of GO-TNA biosensor is also characterized by fluorescence quenching studies as a function of incubation time and GO concentrations. As shown in FIGS. 3A-3B and FIG. 4, the fluorescence signal of Cy3 is significantly quenched within 1 min after 100 nM Cy3TNA-155 are added to the GO suspensions at different concentrations. The fluorescence signal is almost quenched by ≥99.9% after adding 8.8 μL of 0.1 mg/mL GO solution. The interaction between TNA and GO is a complex process influenced by the presence of highly oxidized and hydrophilic domains, as well as carbon-rich hydrophobic domains. These domains exhibit sizes ranging from 2.5 to 6 nm, which are comparable to the length of the currently utilized probes.

The results confirm the successful adsorption of TNAs onto the surface of GO substrates, owing to the interaction between TNA and GO, which involves a combination of TNA base stacking with hydrophobic domains, hydrogen bonding with oxygen-rich domains, and the influence of electrostatic repulsion. These various interactions collectively contribute to the complex adsorption behavior observed under different experimental conditions.

Subsequently, this optimal concentration of GOs to the recognizing probes is used to construct the GO-TNA nanoplatform accordingly.

The working principle of TNA-GO biosensors for miRNA detection is based on the sequence specific recognition via Watson-Crick base pairing. In the absence of target miRNAs, the fluorescence signal of ssTNA probe is very weak for detection due to the quenching effect between GO and ssTNA probe in a close proximity. In the presence of target miRNAs, they interact with the TNA sensing probes to form a duplex TNA-RNA complex followed by dissociation from GO's surface. These binding events separate the GO and fluorophore in a longer distance, resulting in the recovery of its fluorescence signal. The extent of fluorescence enhancement is quantifiably related to the expression level of the target miRNAs.

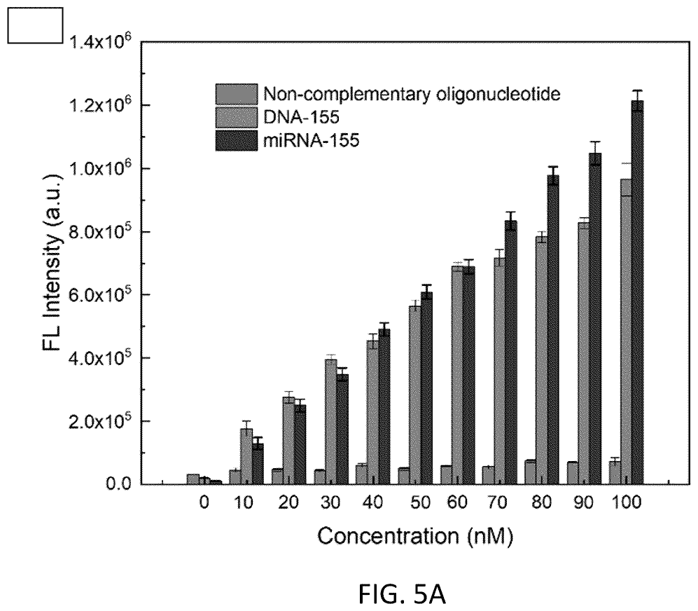

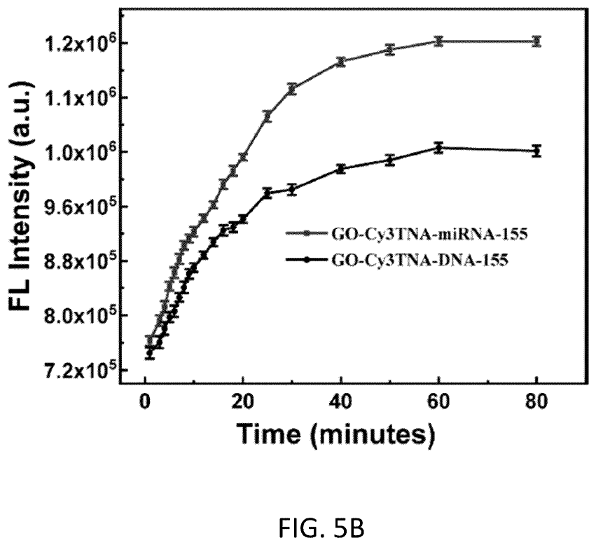

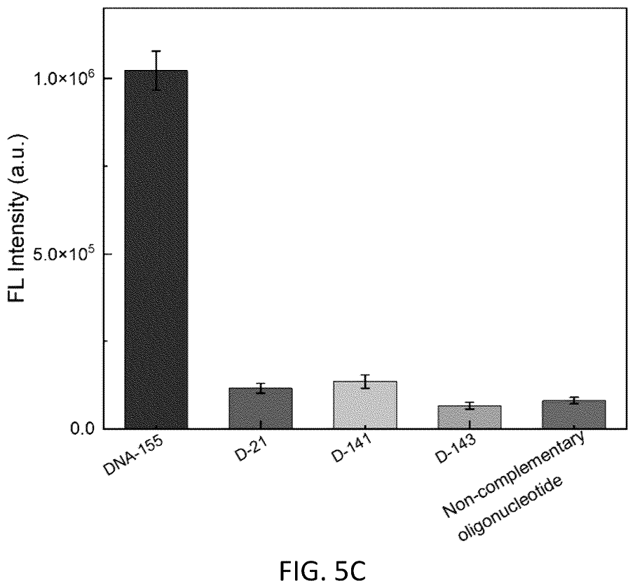

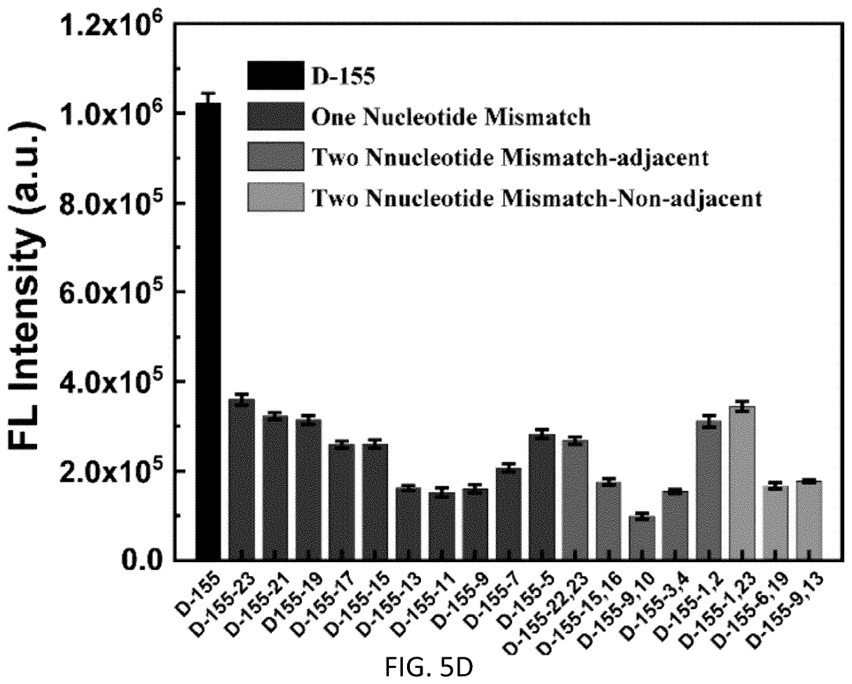

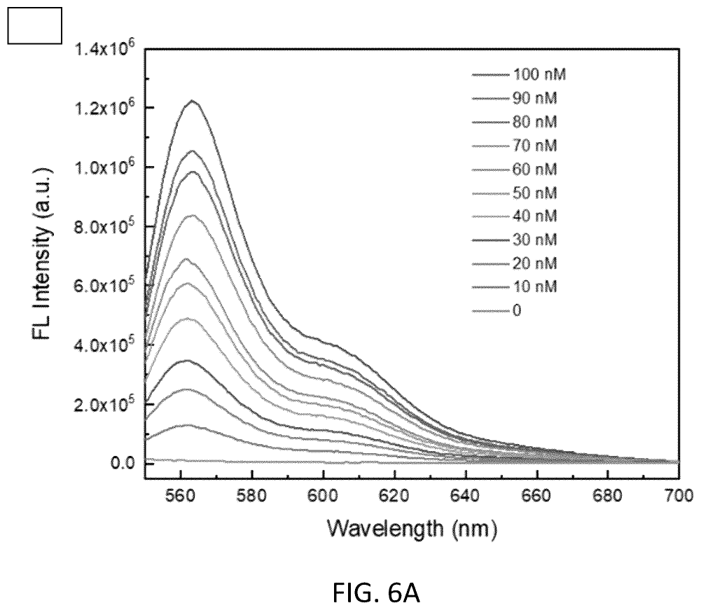

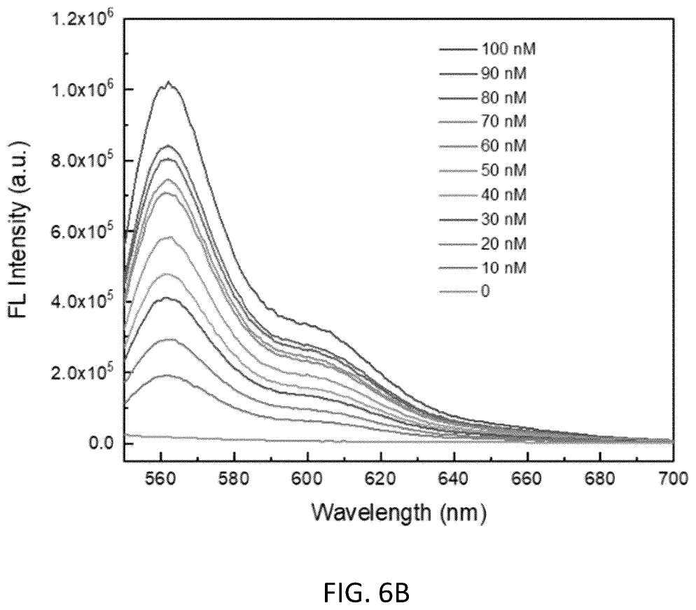

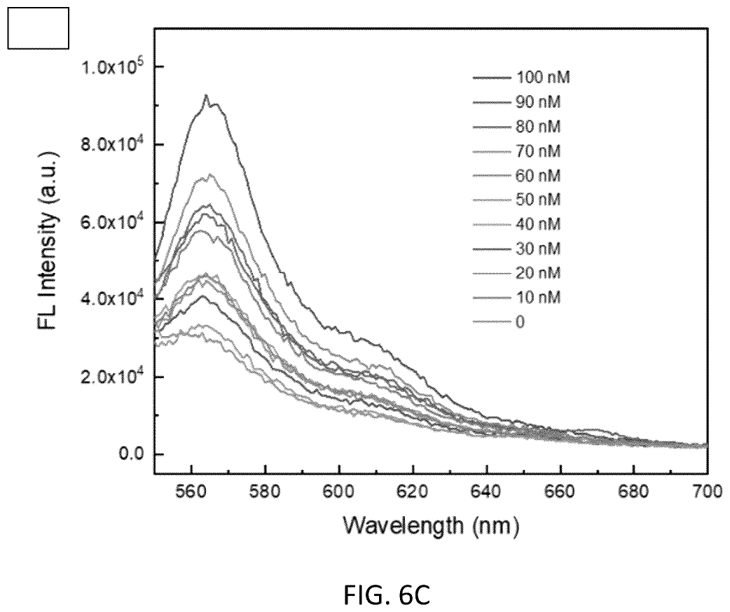

After the optimized GO-Cy3TNA-155 nanoplatform is formed, its detection performance is firstly evaluated by comparing the fluorescence recovery upon addition of its complementary oligonucleotides versus non-complementary oligonucleotides. As shown in FIGS. 5A-5B and FIGS. 6A-6C, after adding the target miRNA-155 or DNA-155, the fluorescence of Cy3 on GO-Cy3TNA-155 is gradually increased as a function of concentrations and incubation time when compared to non-complementary DNAs. This observation is attributed to the sequence specific recognition via Watson-Crick base pairing by forming stable TNA-RNA/DNA hybrid duplexes in high specificity and then followed by their dissociation from the GO's surface, resulting in a “turn-on” fluorescence.

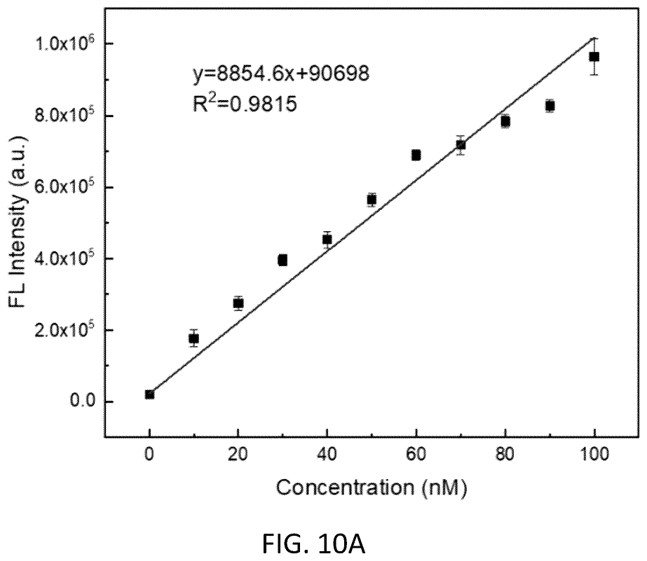

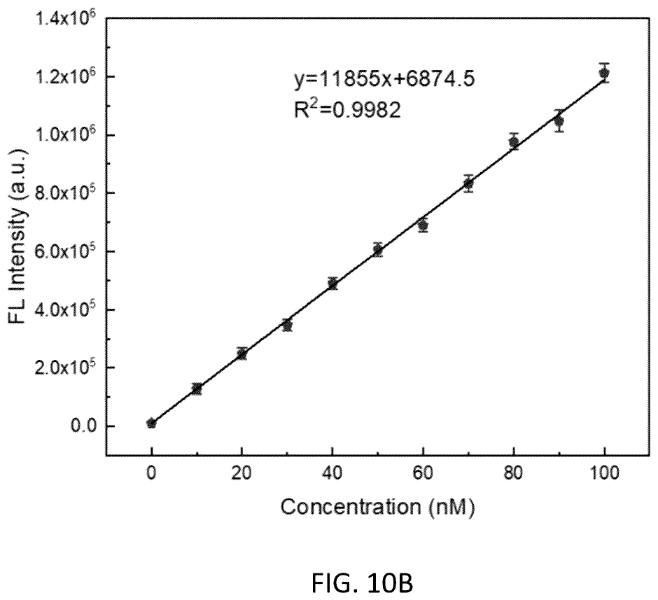

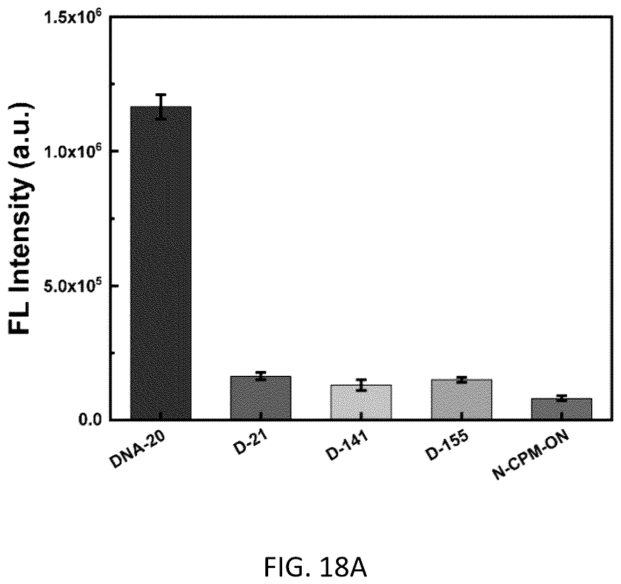

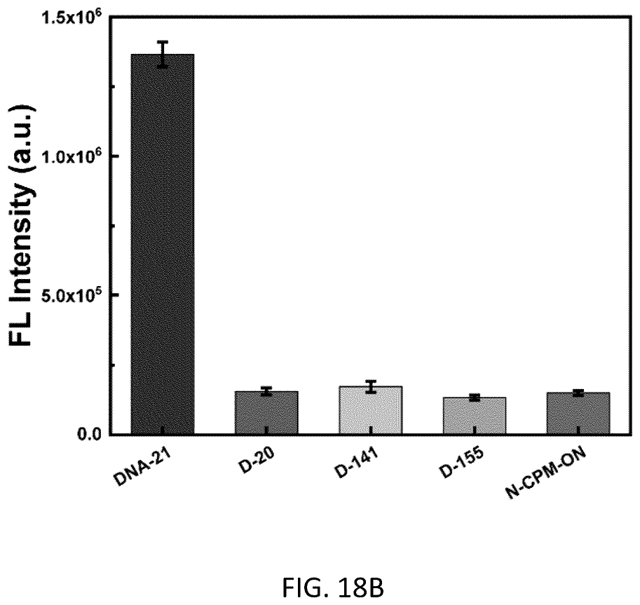

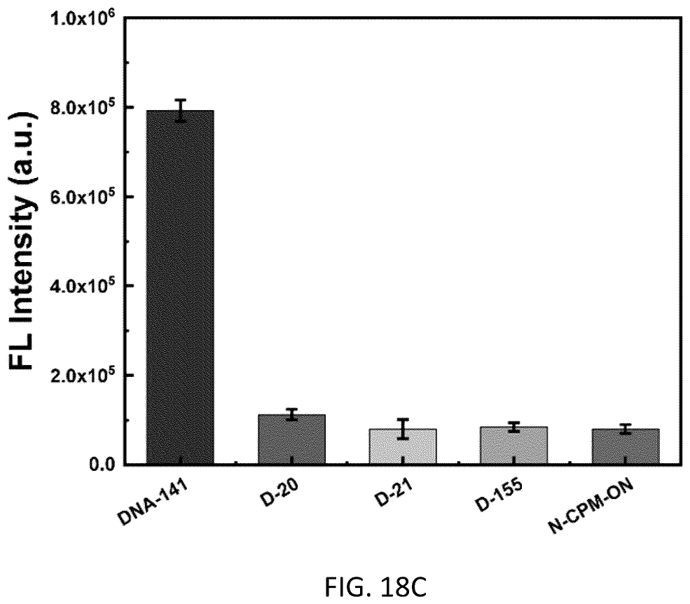

After that, a quantitative detection of target nucleic acids, including DNA and RNA, is also examined by measuring the fluorescence intensity changes of the GO-Cy3TNA platform with increasing the concentration of targets added. The results indicated that good linearity is obtained from 0 nM to 100 nM of target D-155 and miR-155, with a limit of detection (LOD) of 33.88 pM and 28.33 pM, respectively, based on the 3σ rule (FIGS. 10A and 10B). In comparison to the LOD value reported for the previously partial duplexed TNA-based biosensor (2.5 nM), the current GO-TNA sensing platform for nucleic acid detection exhibits a considerably improved LOD of approximately 28.33 pM, representing an ˜88-fold enhancement. The LOD of the GO-TNA system is also comparable to the miRNA detection platform using copper nanocluster, gold nanoparticles, quantum dot, biochip and microarray. Additionally, the LOD of the GO-TNA system is similar to the miRNA detection platform that utilize copper nanocluster and gold nanoparticles. For example, prior studies utilized gold nanoparticles as carriers and achieved an LOD of 70 pM for miRNA-21 detection. Quantum dot-based Förster resonance energy transfer method, achieving LODs of 1.3 nM, 0.9 nM, and 0.7 nM for miRNA-20a, miRNA-20b, and miRNA-21 were employed by other groups, respectively. Another approach utilized a renewable super wettable biochip to detect miRNA-141 with an LOD of 88 pM. Additionally, other groups employed a microsphere-based suspension array, which achieved an LOD of approximately 30 PM for miRNA detection. An additional three TNA probes sere further synthesized, designed to target different miRNAs, specifically miR-20, miR-21, and miR-141. The results obtained, as presented in FIGS. 18A to 18C, demonstrate that all three TNA probes exhibit remarkable specificity in recognizing their respective target miRNAs. Importantly, these detection outcomes serve as mutually independent controls for each other, confirming that TNA probes with specific sequences exclusively identify and bind to their intended targets. This validation underscores the significantly low sequence dependence exhibited by this type of detection probe. Through the testing and validation of multiple TNA capture probes targeting different sequences, the generalizability and reliability of TNA-GO for miRNA detection has been successfully demonstrated. Importantly, the in vitro and in vivo results indicate that even if a small portion of the TNA probe is replaced by cellular proteins or enzymes, it will not significantly affect the recognition properties of the GO-TNA detection system. The inclusion of these additional probes not only reinforces the applicability and versatility of this biosensing platform but also enhances its effectiveness in a broader range of miRNA detection applications.

Based on the aforementioned results, the fluorescence recovery of the GO-TNA detection platform upon the addition of miRNA-155 and DNA-155 are compared. Notably, a significantly stronger fluorescence recovery is observed in the miRNA-155 group compared to the DNA-155 group (as depicted in FIGS. 5A and 5B). These findings indicate that this system possesses heightened sensitivity for detecting miRNA as opposed to DNA. However, considering the relative stability and cost-effectiveness of DNA compared to RNA, and for simplicity, DNA is primarily employed as a substitute for RNA in the subsequent solution tests.

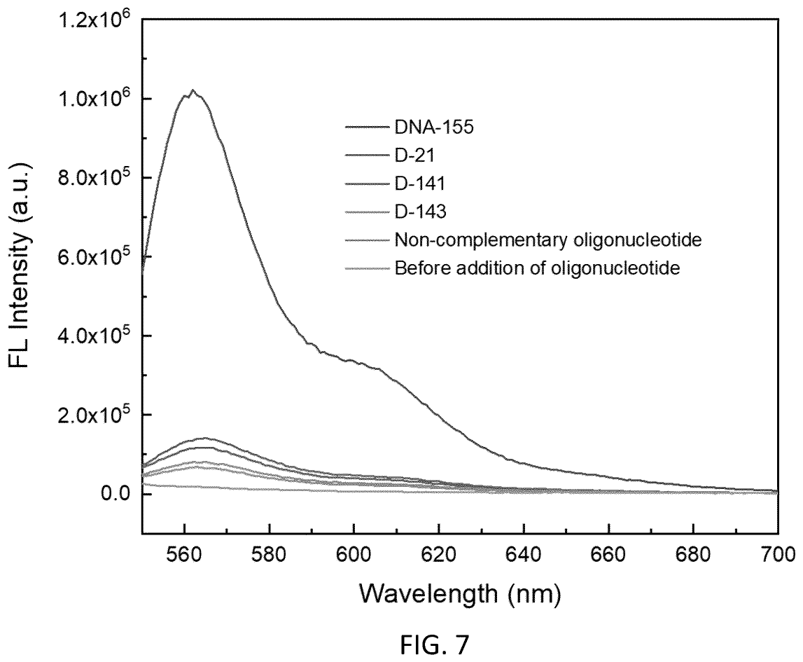

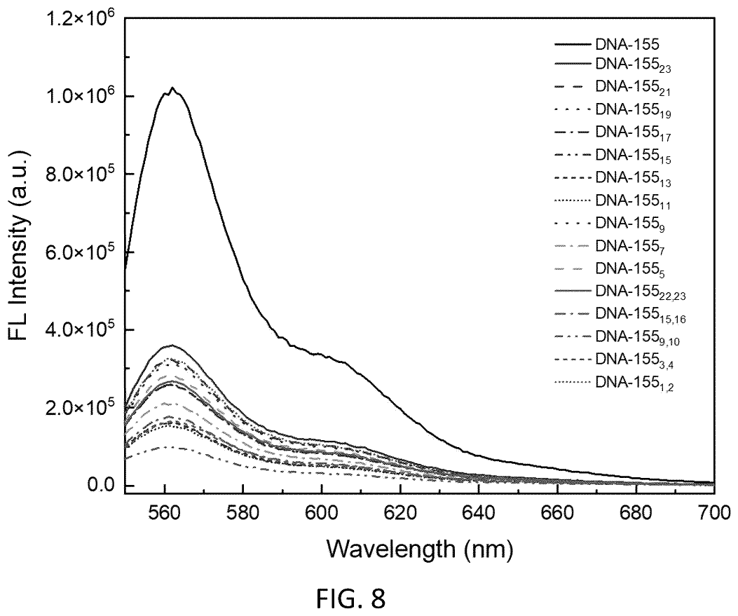

The specificity of the GO-TNA system is also examined by adding other sequences of testing nucleic acids including D-21, D-141 and D-143 which are with the same sequences of miRNA-21, miRNA-141 and miRNA-143 respectively as negative controls. There is no such fluorescence enhancement observed when D-21, D-141 and D-143 are added (FIG. 5C and FIG. 7). Particularly, the fluorescence intensity determined by adding the complementary D-155 is approximately 10-fold increase of that generated by other tested nucleic acids. The high specificity of target detection for identifying tenuous mismatches is of great importance for better understanding of the functions of individual miRNAs in biological systems as there is only one or two nucleotide(s) differences in some miRNA isoforms. The tested oligonucleotides which have the same length are selected, but sequences differ by only one or two nucleotides at different positions to the corresponding target miRNAs. It is found that there is no significant change in the fluorescence signals after adding the oligonucleotides with single nucleotide mismatch when compared to target nucleic acid (FIG. 5D and FIG. 8). Therefore, it is suggested that this GO-TNA system is sequence-specific and sequence-selective for effective single nucleotide recognition.

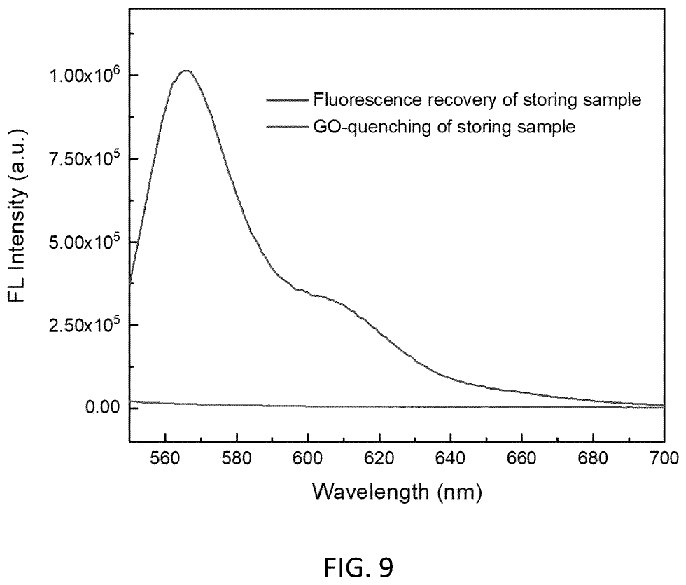

Furthermore, GO-TNA platform maintains its detection effectiveness even after storing at room temperature for half a year, confirming its stability (FIG. 9). A quantitative detection of target nucleic acids including DNA and RNA will also be examined by measuring the fluorescence intensity changes of the GO-Cy3TNA platform with increasing the concentration of targets added. The results indicated that a good linearity is obtained from 0 nM to 100 nM of target D-155 and miR-155 with limit of detection (LOD) of 33.8±0.15 pM and 28.33±0.12 pM respectively based on the 36 rule (FIG. 10). Compared to the reported partial duplexed TNA-based biosensor, these LOD values in pM scale confirmed the suitability of using this GO-TNA platform for intracellular miRNA detection.

The present invention can also be modified with tumor targeting ligands for biodistribution analysis and dynamic real-time imaging/monitoring of target miRNA expressions which are associated with the stages of disease genesis in living organisms. Ny simply conjugating different fluorescence resonance energy transfer (FRET) pairs or fluorescent dye quencher pairs onto the invention, this invention is capable of producing distinct fluorescence signals upon the detection of their corresponding miRNA targets and offer multiplex detection in tested samples. The abnormal miRNA expression and distribution are also closely associated with cancer types and stages, tumor response to treatments, and cellular conditions of differentiating stem cells, thus detecting and visualizing different miRNA expressions in living systems is crucial in effectively investigating miRNA-regulated signaling pathways for anticancer therapy. Northern blot, real-time polymerase chain reaction and microarrays are the most commonly used miRNA detection methods which are applicable only in homogenous solutions and in cell lysates, however they have relatively low sensitivity and throughput, time consuming, high experimental costs and analytical complexity. Although rolling-circle amplification, ligase chain reactions and isothermal amplification have been explored to achieve better sensitivity, they are still not satisfactory for in vivo applications, particularly for monitoring the dynamic expression and distribution of miRNAs in living systems. Thus, the development of a rapid, convenient, cost-effective and sensitive approach for in situ detection of miRNA expression is imperative.

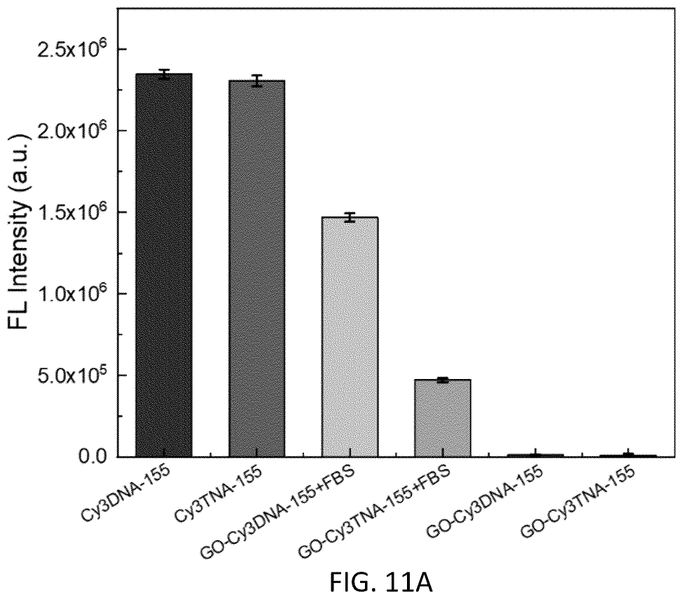

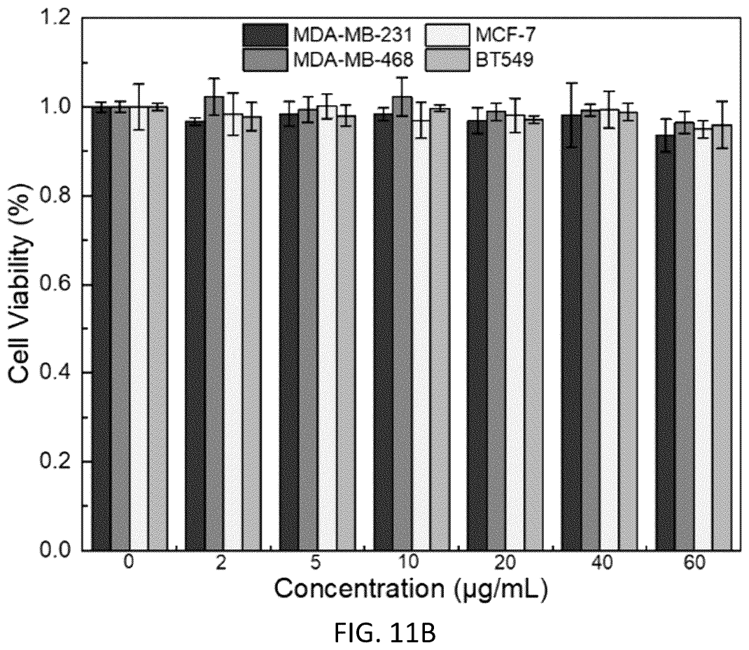

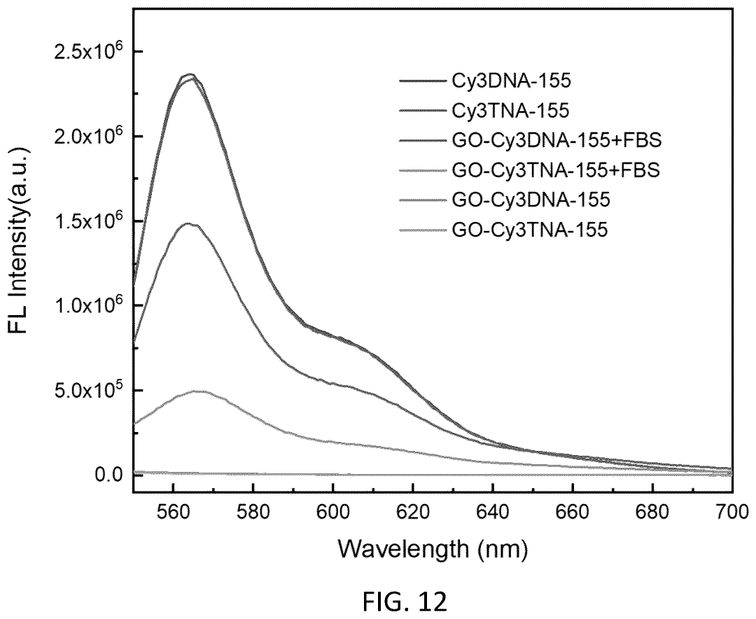

High biocompatibility of the detection tool is of vital importance for in situ imaging and nucleic acid targeting in live cells. The biostability and cytotoxicity of the GO-TNA platform is fully investigated. Firstly, the integrity of the detection platform immobilized with cither TNAs or DNAs as capture probes after fetal bovine serum treatment is tested (FIG. 11A and FIG. 12). Samples are incubated with 10% FBS in 1×TAMg buffer under 8 h at 37° C. Before FBS treatment, the fluorescence intensity of Cy3 in both GO-Cy3TNA-155 and GO-Cy3DNA-155 systems is almost quenched. After FBS treatment, the fluorescence recovery of GO-Cy3DNA-155 systems is significantly increased by ˜3-fold to that of GO-Cy3TNA-155 systems. These results are highly reproducible, evidencing the thorough resistance of the GO-based detection systems with TNA sensing probes to biological degradation when compared to those with DNA capture probes.

This evidence also demonstrates that, under physiological conditions, the GO-Cy3TNA-155 systems exhibits a background signal that is at least three times lower than that of the GO-Cy3DNA-155 system. This finding further confirms the suitability of the detection system for intracellular and in vivo applications. With reduced background signal, the TNA-based platform offers enhanced sensitivity and specificity, enabling accurate and reliable detection of tar-get molecules within living cells and organisms.

In one embodiment, sequences of the probes and tested oligonucleotides are listed in Table 1. The nucleotide mismatch at different positions is shown in bold.

TABLE 1

| Type | Name of oligonucleotide | Sequence(s) |
|---|---|---|
| TNA | Cy3TNA-155 | 3′-ACC CCT ATC ACG ATT AGC ATT |
| | (TNA Capture Probe) | AA-Cy3-2′ |
| | Cy3scrTNAs | 3′-AAA ACC ACG AGC CCT ATT ATC |
| | (TNA Control Probe) | TT-Cy3-2′ |
| RNA | miRNA-155 | 5′-TTA ATG CTA ATC GTG ATA GGG |
| | | GT-3′ |
| DNA | Cy3DNA-155 | 5′-ACC CCT ATC ACG ATT AGC ATT |
| | | AA-Cy3-3′ |
| | DNA-155 | 5′-TTA ATG CTA ATC GTG ATA GGG |
| | | GT-3′ |
| | DNA-155₂₃ | 5′-TTA ATG CTA ATC GTG ATA GGG |
| | | GC-3′ |
| | DNA-155₂₁ | 5′-TTA ATG CTA ATC GTG ATA GGT |
| | | GT-3′ |
| | DNA-155₁₉ | 5′-TTA ATG CTA ATC GTG ATA TGG |
| | | GT-3′ |
| | DNA-155₁₇ | 5′-TTA ATG CTA ATC GTG ACA GGG |
| | | GT-3′ |
| | DNA-155₁₅ | 5′-TTA ATG CTA ATC GTT ATA GGG |
| | | GT-3′ |
| | DNA-155₁₃ | 5′-TTA ATG CTA ATC TTG ATA GGG |
| | | GT-3′ |
| | DNA-155₁₁ | 5′-TTA ATG CTA ACC GTG ATA GGG |
| | | GT-3′ |
| | DNA-155₉ | 5′-TTA ATG CTT ATC GTG ATA GGG |
| | | GT-3′ |
| | DNA-155₇ | 5′-TTA ATG TTA ATC GTG ATA GGG |
| | | GT-3′ |
| | DNA-155₅ | 5′-TTA ACG CTA ATC GTG ATA GGG |
| | | GT-3′ |
| | DNA-155_{22,23} | 5′-TTA ATG CTA ATC GTG ATA GGG |
| | | AC-3′ |
| | DNA-1551_{5,16} | 5′-TTA ATG CTA ATC GTC CTA GGG |
| | | GT-3′ |
| | DNA-155_{9,10} | 5′-TTA ATG CTC CTC GTG ATA GGG |
| | | GT-3′ |
| | DNA-155_{3,4} | 5′-TTC CTG CTA ATC GTG ATA GGG |
| | | GT-3′ |
| | DNA-155_{1,2} | 5′-CCA ATG CTA ATC GTG ATA GGG |
| | | GT-3′ |
| | Non-complementary | 5′-TAT TGT GGA TTC GCG AGT TAA |
| | oligonucleotide | GA-3′ |
| | D-21 | 5′-TAG CTT ATC AGA CTG ATG TTG |
| | | A-3′ |
| | D-141 | 5′-TAA CAT TGT CTG GTA AAG ATG |
| | | G-3′ |
| | D-143 | 5′-TGA GAT GAA GCA CTG TAG CTC- |
| | | 3′ |

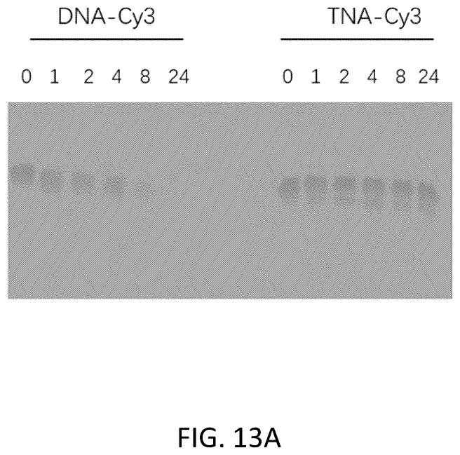

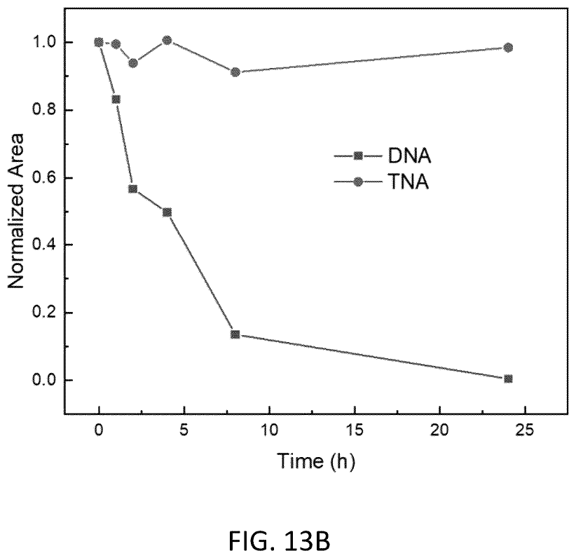

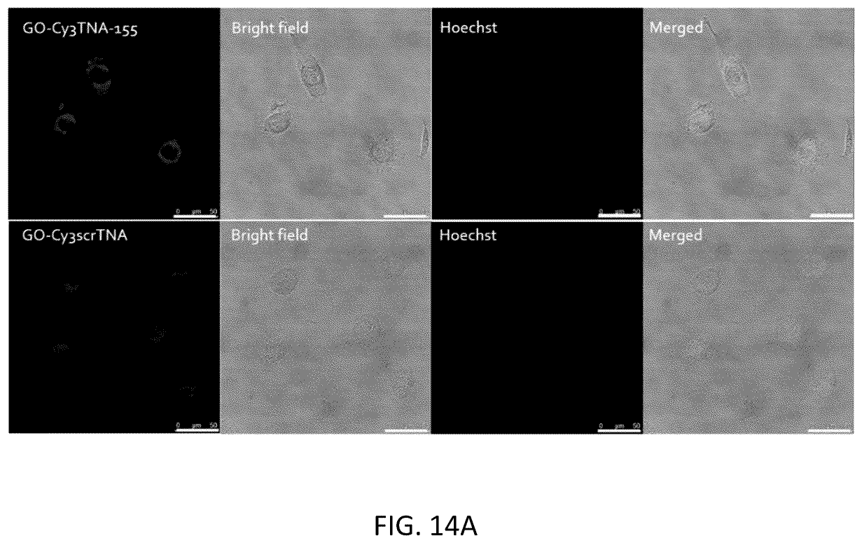

This observation is highly attributed to the two possible phenomena:
1) FBS is a sophisticated blend comprising various biomolecules, including proteins, and nucleases. Previous works have demonstrated that proteins and nucleases possess the ability to partially displace nucleic acid strands from the surface of GO. However, the competitive desorption from proteins in serum is weaker in GO-TNA systems as TNAs have a stronger binding affinity to the GO's surface than DNAs;
2) a relatively less amount of TNA is digested and dissociated from the GO's surface due to its very strong degradation resistance towards FBS when compared to natural nucleic acids (FIG. 13).

Additionally, MTT assay is used to assess the cytotoxicity of GO-TNA platforms. As shown in FIG. 11B, negligible cytotoxicity is observed in four cell lines including MCF-7, MDA-MB-231, MDA-MB-468 and BT-549 cells. The relative cell viability remains to be above 98% and cells are in good shape and health even after incubated at concentration up to 60 μg/mL for 24 h. This confirms the biocompatibility of GO-TNA platforms for long-term intracellular miRNA detection.

Prior investigation into the internalization pathway of GO in HepG2 human liver cancer cells using flow cytometry demonstrated that GO primarily enters HepG2 cells through macropinocytosis and clathrin-dependent endocytosis. Additionally, it is also reported that GO enters cells predominantly via clathrin-dependent endocytosis and to a certain extent through caveolin-dependent endocytosis. It is also previously demonstrated that the TNA enters cells through a temperature-and energy-dependent endocytotic pathway. Based on the collective evidence, it is assumed that clathrin-dependent endocytosis is the prominent mechanism for the internalization of GO-TNA biosensors into cells.

For cellular applicability, confocal fluorescence imaging of three living cells such as BT549, MCF-7 and MDA-MB-468 after treating with GO-Cy3TNA-155 and GO-Cy3ScrTNA systems in PBS-containing culture medium for 12 h is conducted and compared.

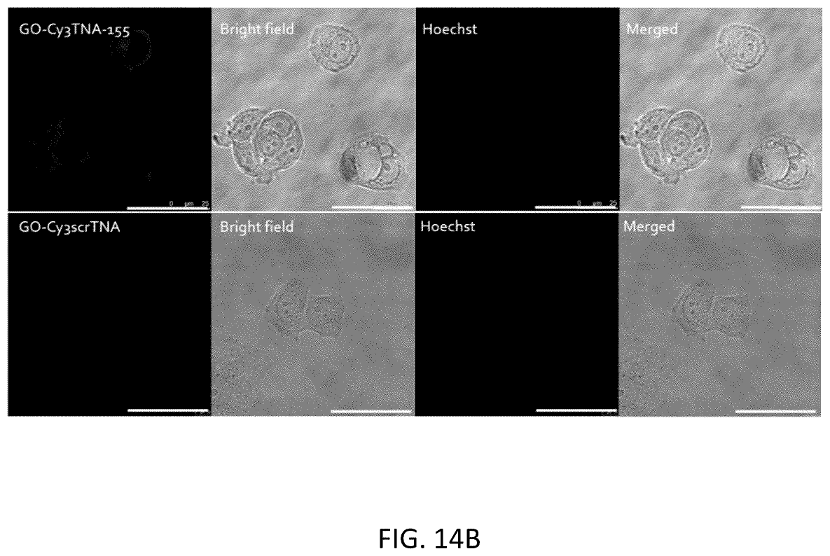

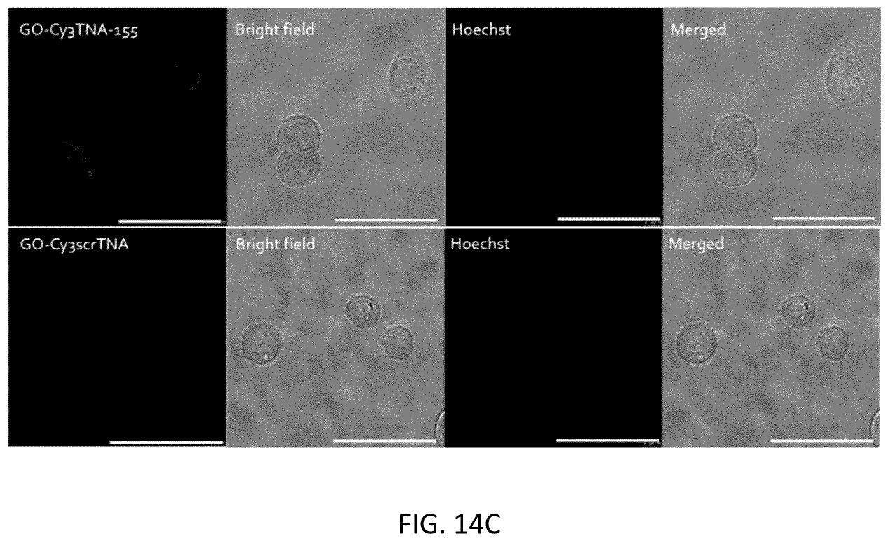

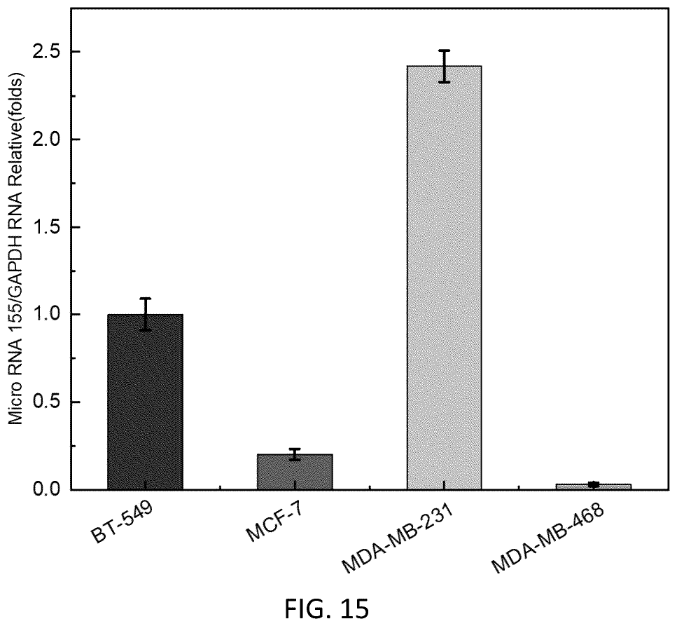

BT549 cells treated with GO-Cy3TNA155 showed bright Cy3 fluorescence in the cytoplasm (FIG. 14A). Compared to BT549, MCF-7 and MDA-MB-468 cells treated with GO-Cy3TNA155 exhibited relatively weaker fluorescence intensity of Cy3 (FIGS. 14B-C). The differences are attributed to the relatively low intrinsic expression level of miR-155 in MCF-7 and MDA-MB-468 than in BT549 as confirmed by quantitative polymerase chain reaction (qPCR) studies (FIG. 15). In contrast, no noticeable fluorescence is observed from GO-Cy3ScrTNA-treated cells.

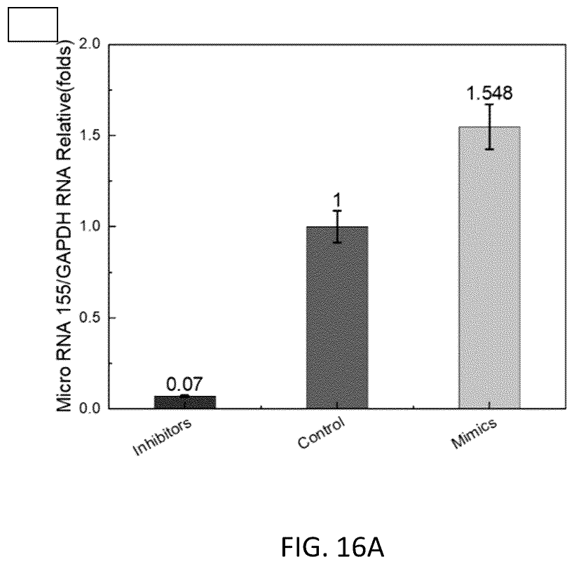

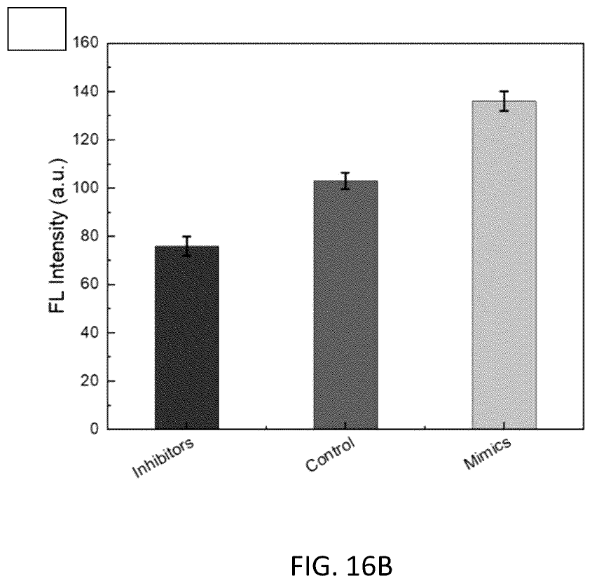

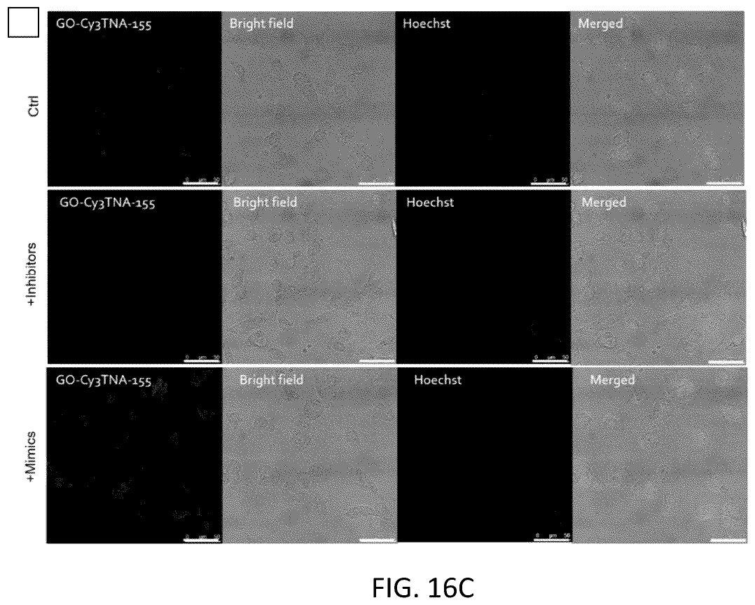

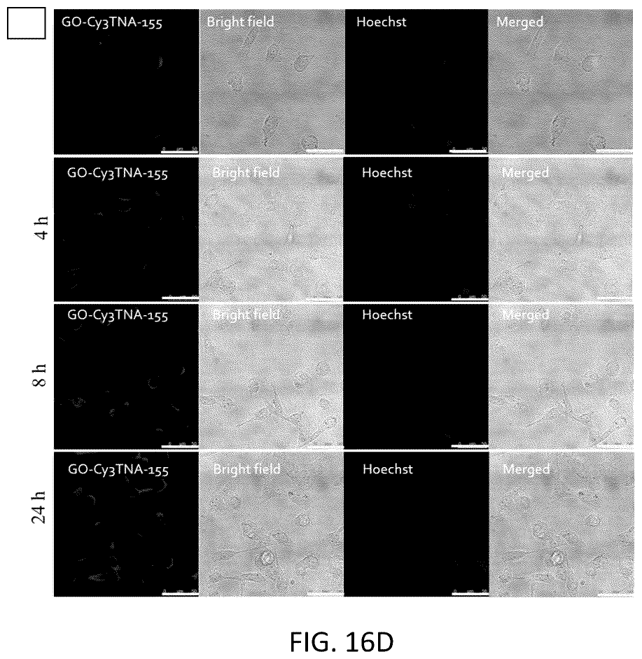

For live-cell applications, accurate monitoring of dynamic change of target nucleic acids such as miRNAs in various cellular states is of great importance during cell and disease developments. Thus, the change of target miR-155 expression levels in miRNA-155 inhibitor treated and miRNA-155 mimic-treated MDA-MB-231 cells is monitored via RT-qPCR, flow cytometry and fluorescence microscopy. The cells are first incubated with the inhibitor or mimic for 24 h to inhibit or promote miR-155 expression respectively before treating with GO-TNA platforms (FIG. 16A). The results show that about 1.5-folds increase of miRNA-155 expression levels in mimics-treated MDA-MB-231 cells is determined. In contrast, a ˜15-fold decrease of miRNA-155 expression levels in miR-155 inhibitor-treated MDA-MB-231 cells is achieved. These results indicate that mature miRNA-155 levels can be altered by adding miRNA mimics and inhibitors.

Subsequently, MDA-MB-231 cell samples treated with mimics and inhibitors are employed to conduct flow cytometry and confocal fluorescence imaging. As shown in FIG. 16B, the fluorescence corresponding to Cy3 probe in GO-Cy3TNA155 detection platform significantly increased inside cells when miRNA-155 mimics is added. In contrast, its fluorescence decreased when miRNA-155 inhibitor is added. These flow cytometry results align with the results of confocal fluorescence studies, confirming the detection platform can relatively reflect the level of miRNA-155 contents in living cells (FIG. 16C).

While the overall trend is consistent between RT-qPCR and flow cytometry studies, the magnitude of decrease caused by inhibitor treatment varied greatly. Initially, the TNA probes are physically adsorbed onto the surface of GO, which also possesses an inherent affinity for proteins or enzymes. Therefore, when GO-loaded TNA enters cells, a fraction of TNA-Cy3 may become displaced from the GO surface by cellular proteins or enzymes. This displacement leads to the presence of fluorescence signals within the cells. Hence, even in cells treated with inhibitors that significantly reduce the levels of miRNA, the presence of cellular proteins or enzymes can still displace TNA-Cy3 from the GO surface, resulting in higher fluorescence signals emitted by the cells compared to the miRNA levels detected by RT-qPCR.

Furthermore, the dynamic change of miRNA-155 expression level in the miRNA-155 inhibitor-treated MDA-MB-231 cells is also monitored as a function of incubation time from 0 h (no inhibitor added) to 8 h. The fluorescence intensity of Cy3 in GO-Cy3TNA155 platform increases in a time-dependent manner (FIG. 16D). These results indicate that GO-TNA platform can monitor cellular miRNA dynamics accurately and conveniently.

Although there is gradual progress of nucleic acid-based biosensors in the area of in vivo imaging, the poor biostability of nucleic acid probes still pose a great challenge for in vivo application in complex environment of the living system. Specifically, the enzymatic degradation and metabolism of natural nucleic acids in blood and in different organs respectively make the half-life of natural nucleic acid-based biosensor very short, limiting its accuracy and ability of continuous monitoring of targets.

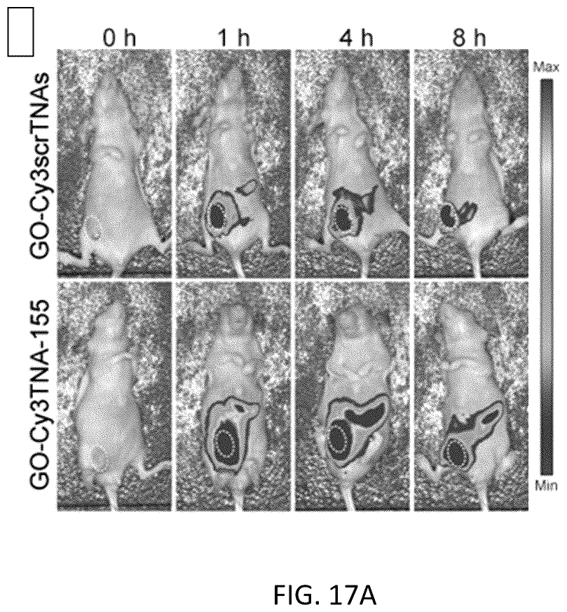

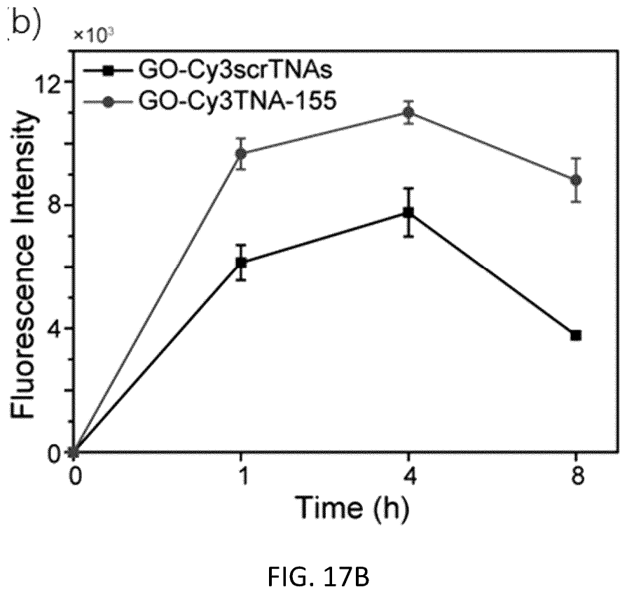

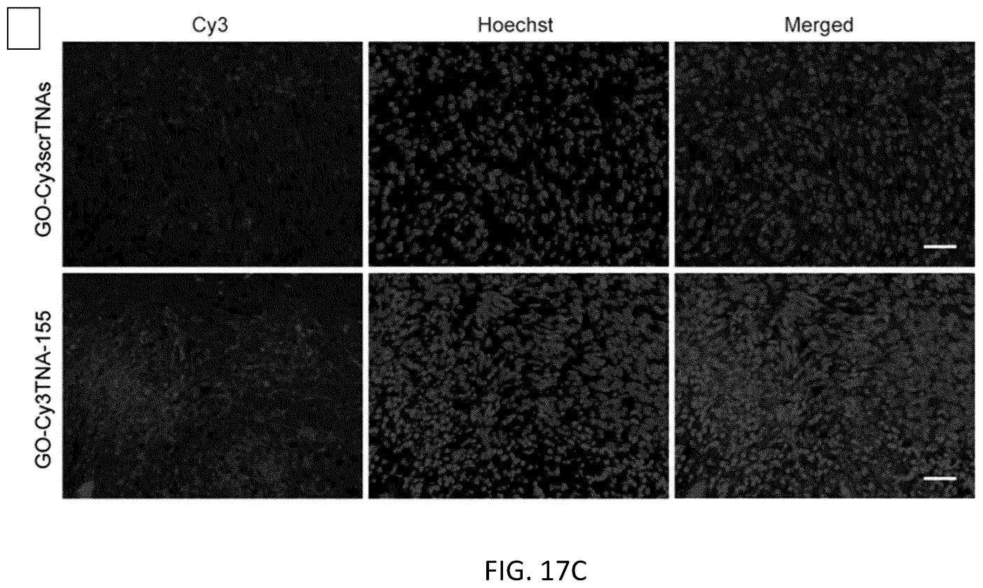

The practicability of the developed GO-TNA biosensor is further assessed to detect and image target nucleic acids in vivo. The MDA-MB-231 tumor-bearing mice models are established and separately treated with GO-Cy3TNA155 as a sample group and GO-Cy3scrTNA as a control group. As shown in FIGS. 17A-17B, the tumor site that receives GO-Cy3TNA155 injection exhibits relatively higher Cy3 signal intensities than that of GO-Cy3scrTNA control groups. The results indicated that the designed GO-TNA biosensors are able to visually detect the target miRNAs in tumor tissues.

In addition, the Cy3 signal intensity reaches the highest level after intratumoral injection of GO-Cy3TNA155 for 4 h, which is basically consistent with the confocal fluorescence imaging results in living cells. The tumors from both GO-TNA nanoplatform-injected groups and analyzed the tumor tissue sections are collected via confocal fluorescence imaging. As expected, the tumor slices from GO-Cy3TNA155-treated mice revealed significantly higher Cy3 fluorescence intensity than the control group (FIG. 17C), indicating the practicality of the GO-TNA biosensor for in vivo nucleic acid detection and imaging. Importantly, the in vitro and in vivo results indicated that straightforward replacing the natural nucleic acids in the current functional TNAs did not abolish their recognition ability. Indeed, success in imaging target miRNAs at in vivo level significantly facilitates the applications of biosensors composed of unnatural nucleic acid probes in the clinic and even for personalized medicine.

While DNA-GO biosensing is a commonly used approach and allows for relatively accurate detection of certain biomolecules under laboratory conditions, it faces a significant challenge when applied in physiological conditions due to the susceptibility of DNA degradation by endogenous nucleases. Instead, TNA exhibits exceptional resistance to degradation even under simulated physiological conditions (FIG. 12) while maintaining capability to form duplexes with complementary DNA and RNA. Specifically, TNA demonstrates higher binding affinity to RNA strands than DNA, which leads to the development of biosensors with enhanced specificity and reliability for miRNA detection. Compared to GO-DNA, the enhanced biological stability observed in TNA-GO is attributed to the specific structural features of TNA. TNA incorporates an unnatural four-carbon threose sugar, which replaces the natural five-carbon ribose sugar found in DNA, while retaining the same nucleobases and phosphodiester bonds. Additionally, TNA lacks the 2′-hydroxyl group present in the threose sugar backbone. Consequently, TNA does not provide recognition sites for nuclease, resulting in much stronger resistance to degradation. This increased resistance to enzymatic degradation contributes to the notable biological stability exhibited by TNA-GO. This limited enzyme adaptation contributes to the higher enzymatic resistance observed for TNA in serum tests. The combination of these factors contributes to the increased stability of TNA assembled on GO compared to DNA-GO biosensors, making it a more favorable choice for long-term imaging or detection in biological environments.

### EXAMPLE

### Regents and Materials

N^{2,9}-diacetylguanine, diphenylcarbamic chloride, 1-Ascorbic acid, silver triflate, 4,4′-dimethoxytrityl chloride, N,O-bis (trimethylsilyl) acetamide, N⁴-benzoylcytosinc, trimethylsilyl trifluoromethanesulfonate, magnesium sulfate, adeninc, thyminc, tertbutyldiphenylchlorosilane, anhydrous oxalic acid, tetrabutylammonium fluoride [which is dissolved in tetrahydrofuran (THF), and the concentration is 1.0 M, 4-dimethylaminopyridine, benzoyl chloride, calcium carbonate, para-toluenesulfonic acid monohydrate, 3-hydroxypropanenitrile, tricthylamine, imidazole, and 2,4,6-trimethylpyridine are ordered from J&K in China. Activated carbon Darco G-60, ammonium persulfate, urea, glycerol, formamide, ammonium chloride, acetic acid, magnesium chloride hexahydrate, StainsAll, (3-aminopropyl) trimethoxysilane, bis (diisopropylamino) chlorophosphine, sodium hydroxide, di-isobutylaluminum hydride solution, serum from human male AB plasma, boric acid, tris (hydroxymethyl) aminomethane, acetic anhydride, ethylenediaminetetraacetic acid (EDTA) disodium salt dehydrate, N,N,N′,N′-tetramethylethylenediamine, sodium bicarbonate, and CelLytic M are ordered from Sigma-Aldrich. Acrylamide/bis-acrylamide (19:1, 40%) is purchased from Bio-Rad. Controlled pore glass (CPG) support with loading densities of 25-40 μmol/g and 1-[(2-cyanocthyl)-(N,N-diisopropyl)]-phosphoramidite are purchased from BioAutomation. The reagents used for solid DNA synthesis are ordered from BioAutomation. Scphadex G-25 (super fine DNA grade) is ordered from Amersham Biosciences. Organelle trackers/markers, penicillin-streptomycin solution, trypsin, Fetal bovine serum (FBS), phosphate-buffered saline (PBS) and Dulbecco's modified Eagle medium (DMEM) are purchased from Invitrogen. 1×TBE buffer with a pH of 8.2 consists of 1.1 mM EDTA, 90 mM boric acid and Tris. The reagent grade of all chemicals/reagents are purchased from J & K in China. Except anhydrous dichloromethane (DCM) distilled over CaH2, the technical grade of all other solvents is used. The GO is purchased from Jicang Nanotechnology Co., Ltd. which is commonly produced using the Hymmers' method. This method results in the formation of well-defined GO sheets with consistent oxygen functionalization throughout the material.

### Instrumentation

Fluorescence studies are performed via a HORIBA Jobin Yvon FluoroMax-4 spectrofluorometer. Fluorescence-activated cell sorting studies are performed using BD FACSCanto™ II flow cytometer. A Leica TCS SP5 laser confocal scanning microscope is used to perform the confocal fluorescence imaging with a magnification of 63. Gel electrophoresis analysis is conducted on Maxi Vertical electrophoresis apparatus with acrylamide of 20 cm×20 cm. Gel scanning is performed via a Fujifilm FLA-9000 scanner. The DNA strands are synthesized on a MerMade MM6 DNA synthesizer (BioAutomation). The 3-(4,5-dimethylthiazol-2-yl)-2,5-diphenyltetrazolium bromide (MTT) studies are performed in a Bio Tek Powerwave XS microplate reader.

### Synthesis of Sequence-Designed TNAs

Sequence-designed TNA oligonucleotides are synthesized on an automatic nucleic acid synthesizer according to the well-established solid-phase synthetic protocols. After that, the TNA strands are purified via denaturing polyacrylamide gel electrophoresis (PAGE) in 1 xTBE buffer with a current of 30 mA for 1.5 h at room temperature. MALDI-TOF analysis is conducted to confirm the successful synthesis of our TNA polymers. The quantification of the synthesized TNA strands is performed by UV-vis analysis.

### Construction of Synthetic GO-TNA naNnoplatform

The sample preparation process involves combining an appropriate amount of HCl-Tris buffer with TNA-Cy3. This mixture results in a final concentration of 100 nM, with a total sample volume of approximately 200 μL. The fluorescence intensity of the prepared sample is measured using a FluorMax instrument. Subsequently, incremental volumes of GO dispersion, specifically 1, 2, 3, 4, 5 μL, with a concentration of 0.1 mg/mL, are gradually added to the sample. As the volume of GO increases, the fluorescence intensity exhibits a predominantly linear or near-linear decrease. However, after reaching a certain threshold, further addition of GO does not significantly affect the fluorescence intensity, which suggests that the added GO reaches a saturation point. After optimizing the best condition, Tris-HCl buffer (171.2 μL, 20 mM, containing 5 mM MgCl2, 5 mM KCl, and 100 mM NaCl at pH 7.4) is mixed with 20 μL of 1 μM TNA-Cy3 in a test tube. Subsequently, GO (8.8 μL) solution at 0.1 g/mL is added to the tube and mixed well before further studies. For all fluorescence tests, the fluorescence intensity of the Cy3 probe is excited at 532 nm and its fluorescence emission is collected at 562 nm.

### Fluorescence Quenching

171.2 μL of 20 mM Tris-HCl buffer is mixed with 20 μL of 1 μM TNA-Cy3 in a test tube for fluorescence measurement. Subsequently, 8.8 μL of GO solution at 0.1 g/mL is added to the tube, mixed well, and measured the fluorescence intensity of Cy3 at 562 nm at different time points including at 0, 5, 10, 15, 20, 25, 30 min.

### Detection and Recovery Tests of TNA-Cy3-GO Platform

171.2 μL of 20 mM Tris-HCl buffer is mixed with 20 μL of 1 μM TNA-Cy3 in a test tube. 8.8 μL of GO solution at 0.1 g/mL is then added to the tube and mixed well to ensure complete quenching prior to fluorescence measurement. To optimize the detection capacity, the recovered fluorescence intensity of Cy3 is determined at different time points (0, 10, 20, 30, 40, 50, 60, 70, 80, 90 min) after addition of 20 μL of 1 μM miRNA-155. Additionally, the recovered fluorescence intensity of Cy3 is also measured after addition of different volumes of 1 μM miRNA 155 for 30 min.

### Specificity and Selectivity Tests

After GO-TNA nanocomplexes are formed in test tubes, 20 μL of 1 μM miRNA-155, D-21, D-141, D-143, scramble and/or 1/2-Mismatched oligonucleotides are added to corresponding tubes. The recovered fluorescence intensity of Cy3 is measured accordingly.

### Cytotoxicity Assay of GO-TNA Nanocomplexes

Cells including MDA-MB-231, MDA-MB-468, BT-549 and MCF-7 are seeded into 96-well microtiter plates (10,000 cells/well) at 37° C. for 24 h, changed the medium for the next steps. Then, different concentrations of GO (0, 2, 5, 10, 20, 40 and 60 μg/mL) are separately added into each well and incubated for additional 24 h. Each test is performed in triplicate. After that, 150 μL of 0.5 mg/mL MTT solution in phosphate-buffered saline (PBS) is added to each well and further incubated for another 4 h. The resulting MTT solution is then removed. 150 μL of dimethyl sulfoxide (DMSO) is then added to each well and incubated for further 30 min. Finally, the absorbance at 490 is measured by the microplate reader.

### Cell Culture

MDA-MB-231, MDA-MB-468, MCF-7 are cultured in DMEM medium containing 100 units/mL penicillin and 10% FBS; BT-549 is cultured in RPMI medium containing 100 units/mL penicillin and 10% FBS. All the cells are cultured at 37° C. with 95% ambient air and 5% CO2 which subjected to a passage every 2 days.

Intracellular miRNA Detection

Cells including MDA-MB-231, MDA-MB-468, BT-549 or MCF-7 are seeded into confocal dishes (29 mm Dish with 10 mm Bottom Well) with the seeding numbers of 100,000/dish. After 24 h incubation, the medium is changed before adding and incubating with the GO-TNA nanocomplexes for further 2 h. During this period prepared several tubes and added 20 μL (10 μM) TNA-Cy3 to each tube, 10 μL (1 mg/mL) GO is added subsequently. After that, 1 mL medium is also added to each tube and mixed well. Then, the mixed solution with TNA-Cy3-GO and medium is added to each confocal dish and incubated for 8 hours. After finishing the incubation, pipetted 1 mL medium from every confocal dish, then added 1 mL medium with the amount of 1 μL (1 mg/mL) Hoechst, incubate for 15 min at 37° C. Then wash the dish for 3 times with PBS. After that added 0.5 mL paraformaldehyde into every dish, incubated for 10-15 min at 37° C. Then, washed the dishes 3 times with PBS. Finally, added 1 mL PBS into each confocal dish for confocal fluorescence imaging.

### Quantitative Real-Time Polymerase Chain Reaction (RT-qPCR)

RT-qPCR is conducted to assess the miRNA 155 level in three different cell lines. Cellular total RNA is extracted using the established protocols provided by the RNeasy Mini Kit. For cDNA synthesis, 1 μg of total RNA is subjected to reverse transcription using the TransScript First-Strand Synthesis SuperMix Kit. Real-time amplification is performed on a QuantStudio 3D Digital PCR system using the TransStart Tip Green qPCR SuperMix, following the manufacturer's instructions. Triplicate PCR reactions (20 μL each) are carried out, starting with an initial denaturation step at 94° C. for 30 s, followed by forty cycles consisting of 5 s at 94° C. and 30 s at 60° C. for each cycle. The primer sequences utilized for miRNA-155 are as follows: the forward strand 5′-GCCGCCTTAATGCTAATCGTGAT-3′ and the reverse strand 5′-ATCCAGTGCAGGGTCCGAGG-3′. Glyceraldehyde-3-phosphate de-hydrogenase (GAPDH) is chosen as an internal control for normalization purposes. Data analysis is performed using the Quant Studio Design & Analysis Software.

### Flow Cytometry

MDA-MB-231, MDA-MB-468, BT-549 and/or MCF-7 are initially cultured in 6-well plate for 24 h. Then, a solution mixture with 20 μL (10 μM) of TNA-Cy3, or scramble TNA-Cy3, 10 μL (1 mg/mL) GO and medium is added to the corresponding confocal dish and further incubated for additional 8 h. After removing the culture medium, cells are digested with 0.2 mL trypsin and further washed three times with PBS. Finally, cell samples are analyzed by a flow cytometer.

### Animals

The Balb/c nude mice (6 weeks) are obtained from SPF Biotechnology Co., Ltd. (Beijing) and housed at the animal center of Affiliated Dongguan Hospital, Southern Medical University in a pathogen-free environment. All animal tests are approved by the Animal Welfare and Ethics Committee, and conducted in accordance with the Guidelines for Institutional Animal Care and Use of Affiliated Dongguan Hospital, Southern Medical University.

### In Vivo miRNA Detection

To start, 5.68 μL Cy3TNA-155 or Cy3scrTNAs (100 μM) is added into 50 μl GO solution (0.5 mg/mL in PBS). The mixture is centrifuged at 3200 rpm/min for 5 min and the supernatant is collected for in vivo testing. The MDA-MB-231 tumor-bearing mice model is established by subcutaneously injecting 1×107 MDA-MB-231 cells (100 82 L in PBS) into right flank of Balb/c nude mice. When the MDA-MB-231 tumor volume reached about 50 mm³, 50 ALGO-Cy3TNA-155 or GO-Cy3scrTNAs is injected into the tumor site. Then the mice are imaged via the Lumina In Vivo Imaging System (IVIS) at different time points (0, 1, 4, and 8 h) after injection to detect the Cy3 fluorescence. The data are quantified using IVIS Image Analysis Software and determined the time point at which the Cy3 signal reached the maximum. At the time point with highest Cy3 signal, the tumor is harvested, sectioned and then analyzed via confocal fluorescence imaging.

### Statistical Analysis

The data presented herein are obtained from at least three independent tests. All results are expressed as mean±standard deviation (SD). Statistical analysis is conducted using Origin software, and differences among multiple groups are assessed using one-way analysis of variance (one-way ANOVA). Statistical significance is defined as *P<0.05.

In conclusion, this present invention demonstrates the first example of stable and reliable detection and imaging of target nucleic acids in vivo using the GO biosensors composed of chemically modified TNA capture probes. Compared to natural DNA probes used in conventional techniques, the GO-TNA biosensor not only enables simple and rapid detection, identification and quantification of target nucleic acids, but also allows to monitor the dynamic change of the expression of target nucleic acids in living cells at different status. Importantly, this GO-TNA biosensor can specifically discriminate single nucleobases mismatch, resulting in high specificity and selectivity. Loading the TNA capture probes onto the GO substrate to form GO-TNA sensing platform for nucleic acid detection shows significant improvement by 1000 times in the detection limit when compared to TNA probes itself. This detection platform has a simple preparation method without expensive and laborious isolation procedures and can be obtained without complicated chemical reactions, which are critical advantages for the real-time analysis of living systems. It is anticipated that success with this stable TNA-based GO sensing nanoplatform will bring new opportunities in the field of disease diagnosis for rapid and accurate detecting and imaging of a variety of disease-related nucleic acid molecules at the in vivo level. Apart from healthcare, they may also lead to potential applications in the areas of environmental analysis and food safety by using different sequences of the TNA-based capture probes for detecting pesticides, antibiotics, or even foodborne pathogenic agents.

On the market, there is only miRCURY® LNA® miRNA Detection Probes on sale from Qiagen (USA). It is designed for specific miRNA detection by in situ hybridization. Some disadvantages of this LNA-based biosensors raised from the several sequence limitations in the synthesis of this nucleic acid analogue. In particular, the design of LNA oligomers is constrained by four requirements: 1) sequences of more than four LNA nucleotides much be circumvented; 2) sequences of three or more Cs or Gs must be prevented; 3) GC content must be kept between 30% and 60%; 4) self-complementarity or cross-hybridization must be avoided. These limitations in the sequences of LNA oligomers that can be synthesized and used as capture probes in biosensors can obviously impair the detection of some mutations in genes of interest. Thus, the commercially available LNA-based probes are not high-throughput biosensors with widespread applicability in biotechnology or in the clinical setting. This invention illuminates for the first time the feasibility of integrating structurally modified unnatural nucleic acid TNAs with GO as a biocompatible and universal biosensor for rapid and dynamic miRNA detection in living systems, offering alternative reliable molecular reagents for miRNA-related diagnostics and therapeutics.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments are chosen and described in order to best explain the principles of the invention and its practical application, thereby enabling others skilled in the art to understand the invention for various embodiments and with various modifications that are suited to the particular use contemplated.

1. A high-specificity, high-selectivity and high-biocompatibility nucleobase mismatch-distinguishing RNA biosensory nanoplatform comprising a threose nucleic acid (TNA) sensing probe and a fluorescence quencher, wherein the TNA sensing probe is nucleobase-engineered to be antisense to a target RNA transcript.

2. The high-specificity, high-selectivity and high-biocompatibility nucleobase mismatch-distinguishing RNA biosensory nanoplatform of claim 1, wherein the TNA sensing probe further comprises a fluorophore tag.

3. The high-specificity, high-selectivity and high-biocompatibility nucleobase mismatch-distinguishing RNA biosensory nanoplatform of claim 2, wherein the fluorophore tag is Cy3 tag.

4. The high-specificity, high-selectivity and high-biocompatibility nucleobase mismatch-distinguishing RNA biosensory nanoplatform of claim 1, wherein the fluorescence quencher is graphite oxide.

5. A method of detecting and/or imaging a target RNA transcript through the high-specificity, high-selectivity and high-biocompatibility nucleobase mismatch-distinguishing RNA biosensory nanoplatform of claim 1, comprising:
synthesizing a TNA sensing probe through nucleobase engineering, such that the TNA sensing probe is antisense to the target RNA transcript;
introducing a fluorophore tag to the TNA sensing probe;
binding the antisense capture sequence of the TNA sensing probe to the target RNA transcript;
forming a double-stranded TNA-RNA duplex structure; and
dissociating the TNA-RNA duplex structure from the fluorescence quencher surface.

6. The method of claim 5, wherein the target RNA transcript further comprises a microRNA miRNA-155.

7. The method of claim 5, wherein the target RNA transcript is an oncogenic microRNA.
